(19) United States
(12) Patent Application Publication
(10) Pub. No.: US 2025/0360109 A1
(43) Pub. Date: Nov. 27, 2025

(54) COMPOSITIONS AND METHODS FOR TREATING DEPRESSION IN FEMALES

(71) Applicant: SIRTSEI PHARMACEUTICALS, INC., Morrisville, NC (US)

(72) Inventors: Gregory Cooksey Rigdon, Wake Forest, NC (US); Kelly J. Abernathy, Cary, NC (US); Michael Friedrich Ackermann, Holly Springs, NC (US); Yuki Prescott, Apex, NC (US); Stephen E. Butts, Apex, NC (US)

(21) Appl. No.: 19/107,324

(22) PCT Filed: Sep. 14, 2023
(86) PCT No.: PCT/US2023/074182

§ 371 Date: Feb. 27, 2025

Related U.S. Application Data
(60) Provisional application No. 63/375,872, filed on Sep. 16, 2022.

(51) Int. Cl. A61K 31/4245; A61P 25/24

(52) U.S. Cl. CPC A61K 31/4245; A61P 25/24

(57) ABSTRACT

The present invention relates to methods of treating depression in human females by administering a SIRT6 activator.

### STATEMENT OF PRIORITY

This application claims the benefit of U.S. Provisional Application Ser. No. 63/375,872, filed Sep. 16, 2022, the entire contents of which are incorporated by reference herein.

### FIELD OF THE INVENTION

The present invention relates to methods of treating depression in human females by administering a SIRT6 activator.

### BACKGROUND OF THE INVENTION

Mood disorders are some of the most common mental illnesses. Depression is a psychological disorder characterized by dramatic decline in both mental and physical conditions. The toll extracted by clinical depression, characterized by a despondent feeling, loss of interest in pleasurable activities, guilt, worthlessness, and trouble concentrating, is of immense medical concern. In the U.S. alone, approximately 16 million people or 7% of the adults are afflicted with major depressive disorder, which may also include abnormalities in appetite and sleep and loss of productivity and suicidal ideation. The actual suicide rate, estimated at 1 million worldwide, not only affects the afflicted individual but also the family and friends and at times the entire community.

Mood disorders can be treated through psychotherapy and medications, such as anti-depressants. Unfortunately, current medications may take weeks to months to achieve their full effects and in the meantime, patients continue to suffer from their symptoms and continue to be at risk. Moreover, side effects from these medications can range from unpleasant to life-threatening; for instance, there can be an increased risk of suicide, hostility, and even homicidal behavior.

There is a need in the art for effective treatments for depression.

### SUMMARY OF THE INVENTION

The present invention is based on the determination that activators of sirtuin 6 (SIRT6) provide a significant therapeutic effect for depression in human females while being ineffective in human males. Without being bound by theory, one possible explanation for the gender distinction is that there are differences in SIRT6 function in females versus males. For example, serum concentrations of SIRT6 enzyme are higher in females than in males (Zhao et al., BMC Geriatrics 21:452 (2021)). In another study, it was found that SIRT6 polymorphism at rs350846 (CC genotype) and rs107251 (TT genotype) were significantly correlated with the occurrence of postpartum depressive symptoms (Luo et al., Neuropysch. Dis. Treatment 16:3225 (2020)). Regardless of the mechanism of action, the present invention advantageously provides gender-specific treatments for depressive disorders.

Thus, one aspect of the invention relates to a method of treating depression in a human female subject in need thereof, comprising administering to the subject a therapeutically effective amount of a SIRT6 activator, thereby treating the depression.

Another aspect of the invention relates to a method of treating depression in a human female subject in need thereof, comprising identifying the subject as female and administering to the identified female subject a therapeutically effective amount of a SIRT6 activator, thereby treating the depression.

A further aspect of the invention relates to a method of treating depression in a human subject in need thereof, comprising:
a) determining the gender of the subject; and
b) administering to the subject a therapeutically effective amount of a SIRT6 activator if the subject is determined to be female, thereby treating the depression, or not administering to the subject a therapeutically effective amount of a SIRT6 activator if the subject is determined to be male.

These and other aspects of the invention are set forth in more detail in the description of the invention below.

### DETAILED DESCRIPTION OF EMBODIMENTS OF THE INVENTION

The present invention is explained in greater detail below. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which do not depart from the instant invention. Hence, the following specification is intended to illustrate some particular embodiments of the invention, and not to exhaustively specify all permutations, combinations and variations thereof.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents, nucleotide sequences, amino acid sequences and other references mentioned herein are incorporated by reference in their entirety.

As used in the description of the invention and the appended claims, the singular forms “a,” “an” and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (“or”).

Moreover, the present invention also contemplates that in some embodiments of the invention, any feature or combination of features set forth herein can be excluded or omitted.

Furthermore, the term “about,” as used herein when referring to a measurable value such as an amount of a compound or agent of this invention, dose, time, temperature, and the like, is meant to encompass variations of ±10%, ±5%, ±1%, ±0.5%, or even ±0.1% of the specified amount.

As used herein, the transitional phrase “consisting essentially of” is to be interpreted as encompassing the recited materials or steps and those that do not materially affect the basic and novel characteristic(s) of the claimed invention. Thus, the term “consisting essentially of” as used herein should not be interpreted as equivalent to “comprising.”

By the term “treat,” “treating,” or “treatment of” (or grammatically equivalent terms) is meant to reduce or to at least partially improve or ameliorate the severity of the subject's condition and/or to alleviate, mitigate or decrease in at least one clinical symptom and/or to delay the progression of the condition.

[As used herein, the term “prevent,” “prevents,” or “prevention” (and grammatical equivalents thereof) means to delay or inhibit the onset of a disease. The terms are not meant to require complete abolition of disease, and encompass any type of prophylactic treatment to reduce the incidence of the condition or delays the onset of the condition.

A “treatment effective” amount as used herein is an amount that is sufficient to provide some improvement or benefit to the subject. Alternatively stated, a “treatment effective” amount is an amount that will provide some alleviation, mitigation, decrease or stabilization in at least one clinical symptom in the subject. Those skilled in the art will appreciate that the therapeutic effects need not be complete or curative, as long as some benefit is provided to the subject.

A “prevention effective” amount as used herein is an amount that is sufficient to prevent and/or delay the onset of a disease, disorder and/or clinical symptoms in a subject and/or to reduce and/or delay the severity of the onset of a disease, disorder and/or clinical symptoms in a subject relative to what would occur in the absence of the methods of the invention. Those skilled in the art will appreciate that the level of prevention need not be complete, as long as some benefit is provided to the subject.

“Pharmaceutically acceptable,” as used herein, means a material that is not biologically or otherwise undesirable, i.e., the material can be administered to an individual along with the compositions of this invention, without causing substantial deleterious biological effects or interacting in a deleterious manner with any of the other components of the composition in which it is contained. The material would naturally be selected to minimize any degradation of the active ingredient and to minimize any adverse side effects in the subject, as would be well known to one of skill in the art (see, e.g., Remington's Pharmaceutical Science; 21^{st} ed. 2005). Exemplary pharmaceutically acceptable carriers for the compositions of this invention include, but are not limited to, sterile pyrogen-free water and sterile pyrogen-free physiological saline solution.

A first aspect of the invention relates to a method of treating depression in a human female subject in need thereof, comprising administering to the subject a therapeutically effective amount of a sirtuin 6 (SIRT6) activator, thereby treating the depression.

Another aspect of the invention relates to a method of treating depression in a human female subject in need thereof, comprising identifying the subject as female and administering to the identified female subject a therapeutically effective amount of a SIRT6 activator, thereby treating the depression.

A further aspect of the invention relates to a method of treating depression in a human subject in need thereof, comprising:
a) determining the gender of the subject; and
b) administering to the subject a therapeutically effective amount of a SIRT6 activator if the subject is determined to be female, thereby treating the depression, or not administering to the subject a therapeutically effective amount of a SIRT6 activator if the subject is determined to be male.

Identifying a subject as female or determining the gender of the subject may be carried out by any method known in the art. In some embodiments, the method comprises determining whether the subject has two X chromosomes, i.e., whether the subject is a genetic female. In other embodiments, the method comprises determining the level of circulating female hormones (e.g., estrogen and progestin) in the subject. If the subject has a level of circulating female hormones that is within the average level in the general population for a female of that age, the subject is considered female.

The methods of the invention may be used to treat or prevent any type of depression. Types of depression include, without limitation, major depressive disorder, persistent depressive disorder, minor depression, treatment-resistant depression, substance/medication-induced depression, depressive disorder secondary to medical illness, perinatal and postpartum depression, premenstrual dysphoric disorder, seasonal affective disorder, psychotic depression, and bipolar disorder.

In some embodiments, treatment of depression using the methods of the invention result in a measurable improvement in depression symptoms, e.g., as measured by standard depression scale questionnaires, e.g., the Montgomery-Asberg Depression Rating Scale (MADRS). In some embodiments, the treatment results in improvements in test scores by at least 1%, 2%, 3%, 4%, 5%, 6%, 7%, 8%, 9%, 10%, 15%, 20%, 30%, 40%, 50%, or more. In some embodiments, the treatment results in improvements in the MADRS total score of at least 1, 2, 3, 4, 5, 6, 7, 8, 9, 10 or more points, e.g., over a 4-week period.

The methods of the invention may be carried out with any SIRT6 activator known in the art or later developed. Examples of SIRT6 activators include, without limitation, quercetin, isoquercetin, kaempferol, luteolin, cyanidin, fisetin, delphinidin, icariin, N-acetylethanolamines, oleic acid, linoleic acid, fucoidan, MDL-800, MDL-811, UBCS038 (You et al., Angew. Chem. Int. Ed. 56:1007 (2017)), UBCS039, UBCS040, UBCS058, UBCS060, UBCS068, myristic acid, OEA, CL5D, 10b, 5-CI-PZA, BHJH-TM3, 15f, 17a (catechin gallate), 19b (OSS_128167), 20b, 21b, 22a (A127-(CONHPr)-B178), and 23. The compounds are described in more detail in Fiorentino et al., J. Med. Chem. 64:9732 (2021) and Akter et al., Int. J. Mol. Sci. 22:4180 (2021), each incorporated by reference herein in its entirety.

A further example of a SIRT6 activator is a compound of Formula 1 or a pharmaceutically acceptable salt thereof:

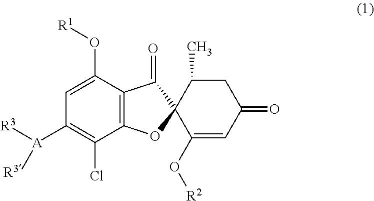

wherein:
R¹ is a C1-C6 alkyl group optionally substituted with the same or different one to two substituents selected from a substituent group X,
a C3-C6 cycloalkyl group optionally substituted with the same or different one to two substituents selected from the substituent group X, or
a 4-7 membered saturated heterocyclic group optionally substituted with the same or different one to two substituents selected from the substituent group X,
R² is a C1-C6 alkyl group optionally substituted with the same or different one to two substituents selected from the substituent group X,
a C3-C6 cycloalkyl group optionally substituted with the same or different one to two substituents selected from the substituent group X, or
a 4-7 membered saturated heterocyclic group optionally substituted with the same or different one to two substituents selected from the substituent group X,
A is a 5-membered aromatic heterocyclic ring,
a 6-membered aromatic heterocyclic ring,
an 8-10 membered condensed aromatic heterocyclic ring,
a 5-7 membered unsaturated heterocyclic ring,
a 4-7 membered saturated heterocyclic ring,
a benzene ring, —CH═, or a cyano group, wherein when A is a cyano group, R³ and R³′ do not exist,
R³ and R³′ are each independently a hydrogen atom, a halogen atom, a cyano group, a hydroxy group, an oxo group,
a C1-C6 alkyl group optionally substituted with the same or different one to two substituents selected from the substituent group X,
a C1-C6 alkoxy group optionally substituted with the same or different one to two substituents selected from the substituent group X,
a C2-C6 alkenyl group optionally substituted with the same or different one to two substituents selected from the substituent group X,
a C2-C6 alkynyl group optionally substituted with the same or different one to two substituents selected from the substituent group X,
a C3-C6 cycloalkyl group optionally substituted with the same or different one to two substituents selected from the substituent group X,
an amino group optionally substituted with the same or different one to two substituents selected from the substituent group X,
a C1-C6 alkoxycarbonyl group optionally substituted with the same or different one to two substituents selected from the substituent group X,
a carbamoyl group optionally substituted with the same or different one to two substituents selected from the substituent group X,
a phenyl group optionally substituted with the same or different one to two substituents selected from the substituent group X,
a 5-membered aromatic heterocyclic group optionally substituted with the same or different one to two substituents selected from the substituent group X,
a 6-membered aromatic heterocyclic group optionally substituted with the same or different one to two substituents selected from the substituent group X,
a 5-7 membered unsaturated heterocyclic group optionally substituted with the same or different one to two substituents selected from the substituent group X,
a 4-7 membered saturated heterocyclic group optionally substituted with the same or different one to two substituents selected from the substituent group X,
an 8-10 membered condensed aromatic heterocyclic group optionally substituted with the same or different one to two substituents selected from the substituent group X, or
R³ and R³′ may form a 5-7 membered unsaturated heterocyclic ring, a 4-7 membered saturated heterocyclic ring, or a C3-C6 cycloalkyl ring as a ring that binds to each other and condenses with A, and the ring is optionally substituted with the same or different one to two substituents selected from the substituent group X,
substituent group X is a halogen atom, a cyano group, a hydroxy group, an oxo group, a C1-C6 alkyl group, a hydroxy C1-C6 alkyl group, a C1-C6 alkoxy C1-C6 alkyl group, a C1-C6 haloalkyl group, a C3-C6 cycloalkyl group, a C3-C6 halocycloalkyl group, a phenyl group optionally substituted with the same or different one to two substituents selected from a substituent group Y,
a 5-membered aromatic heterocyclic group optionally substituted with the same or different one to two substituents selected from the substituent group Y,
a 6-membered aromatic heterocyclic group optionally substituted with the same or different one to two substituents selected from the substituent group Y,
a 4-7 membered saturated heterocyclic group optionally substituted with the same or different one to two substituents selected from the substituent group Y,
a C1-C6 alkoxy group, a C1-C6 haloalkoxy group, a C3-C6 cycloalkoxy group, a C3-C6 halocycloalkoxy group,
a phenoxy group optionally substituted with the same or different one to two substituents selected from the substituent group Y,
a 5-membered aromatic heterocyclic oxy group optionally substituted with the same or different one to two substituents selected from the substituent group Y,
a 6-membered aromatic heterocyclic oxy group optionally substituted with the same or different one to two substituents selected from the substituent group Y,
a 4-7 membered saturated heterocyclic oxy group optionally substituted with the same or different one to two substituents selected from the substituent group Y,
a C1-C6 alkoxycarbonyl group, a C3-C6 cycloalkoxycarbonyl group, a carboxy group, a C1-C6 alkylcarbonyl group, a C3-C6 cycloalkylcarbonyl group,
a phenylcarbonyl group optionally substituted with the same or different one to two substituents selected from the substituent group Y,
a carbamoyl group, a mono (C1-C6 alkyl)aminocarbonyl group, a di (C1-C6 alkyl) aminocarbonyl group, a mono (C1-C6 alkyl)aminosulfonyl group, a di (C1-C6 alkyl) aminosulfonyl group, an amino group, a mono (C1-C6 alkyl) amino group, a di (C1-C6 alkyl) amino group, a C1-C6 alkoxycarbonylamino group, a mono (C1-C6 alkyl)aminocarbonylamino group, a di (C1-C6 alkyl)aminocarbonylamino group, a C1-C6 alkylcarbonylamino group,
a phenylcarbonylamino group optionally substituted with the same or different one to two substituents selected from the substituent group Y,
a 5-membered aromatic heterocyclic carbonylamino group optionally substituted with the same or different one to two substituents selected from the substituent group Y,
a 6-membered aromatic heterocyclic carbonylamino group optionally substituted with the same or different one to two substituents selected from the substituent group Y, or
a C1-C6 alkylsulfonylamino group,
substituent group Y is a C1-C6 alkyl group, a C1-C6 alkoxy group, a halogen atom, or a hydroxy group.

In some embodiments, the compound of Formula 1 is any compound selected from the following group:
(2S,5′R)-7-chloro-6-(5-ethyl-1,3,4-oxadiazol-2-yl)-3′,4-dimethoxy-5′-methyl-spiro[benzofuran-2,4′-cyclohex-2-ene]-1′,3-dione;
(2S,5′R)-7-chloro-3′,4-dimethoxy-5′-methyl-6-(5-tetrahydropyran-4-yl-1,3,4-oxadiazol-2-yl) spiro[benzofuran-2,4′-cyclohex-2-ene]-1′,3-dione;
(2S,5′R)-7-chloro-3′,4-dimethoxy-5′-methyl-6-[5-(1-methyl-4-piperidyl)-1,3,4-oxadiazol-2-yl] spiro[benzofuran-2,4′-cyclohex-2-ene]-1′,3-dione;
(2S,5′R)-7-chloro-6-[5-(4-fluoro-1-methyl-4-piperidyl)-1,3,4-oxadiazol-2-yl]-3′,4-dimethoxy-5′-methyl-spiro[benzofuran-2,4′-cyclohex-2-ene]-1′,3-dione;
(2S,5′R)-7-chloro-3′,4-dimethoxy-6-[5-[(1S)-1-methoxyethyl]-1,3,4-oxadiazol-2-yl]-5′-methyl-spiro[benzofuran-2,4′-cyclohex-2-ene]-1′,3-dione;
(2S,5′R)-7-chloro-4-ethoxy-3′-methoxy-5′-methyl-6-(5-methyl-1,3,4-oxadiazol-2-yl) spiro[benzofuran-2,4′-cyclohex-2-ene]-1′,3-dione;
(2S,5′R)-7-chloro-4-(difluoromethoxy)-3′-methoxy-5′-methyl-6-(5-methyl-1,3,4-oxadiazol-2-yl) spiro[benzofuran-2,4′-cyclohex-2-ene]-1′,3-dione;
(2S,5′R)-7-chloro-3′,4-dimethoxy-6-[3-(1-methoxyethyl)-1,2,4-oxadiazol-5-yl]-5′-methyl-spiro[benzofuran-2,4′-cyclohex-2-ene]-1′,3-dione;
(2S,5′R)-7-chloro-6-[3-(1-hydroxy-1-methyl-ethyl)-1,2,4-oxadiazol-5-yl]-3′,4-dimethoxy-5′-met hyl-spiro[benzofuran-2,4′-cyclohex-2-ene]-1′,3-dione;
(2S,5′R)-7-chloro-3′,4-dimethoxy-5′-methyl-6-(1H-pyrazol-5-yl) spiro[benzofuran-2,4′-cyclohex-2-ene]-1′,3-dione;
(2S,5′R)-7-chloro-3′,4-dimethoxy-6-[1-(2-methoxyethyl) pyrazol-3-yl]-5′-methyl-spiro[benzofuran-2,4′-cyclohex-2-ene]-1′,3-dione;
(2S,5′R)-7-chloro-6-(1,8-dioxa-2-azaspiro[4.5]dec-2-en-3-yl)-3′,4-dimethoxy-5′-methyl-spiro[benzofuran-2,4′-cyclohex-2-ene]-1′,3-dione;
(2S,5′R)-7-chloro-3′,4-dimethoxy-5′-methyl-6-(8-methyl-1-oxa-2,8-diazaspiro[4.5]dec-2-en-3-yl) spiro[benzofuran-2,4′-cyclohex-2-ene]-1′,3-dione;
(2S,5′R)-7-chloro-3′,4-dimethoxy-6-(2-methoxypyrimidin-5-yl)-5′-methyl-spiro[benzofuran-2,4′-cyclohex-2-ene]-1′,3-dione;
(2S,5′R)-7-chloro-3′,4-dimethoxy-6-(6-methoxy-3-pyridyl)-5′-methyl-spiro[benzofuran-2,4′-cyclohex-2-ene]-1′,3-dione; or
(2S,5′R)-7-chloro-3′,4-dimethoxy-5′-methyl-6-(3-pyridyl) spiro[benzofuran-2,4′-cyclohex-2-ene]-1′,3-dione.

In some embodiments, the compound of Formula 1 is a compound of Formula 1′ or a pharmacologically acceptable salt thereof:

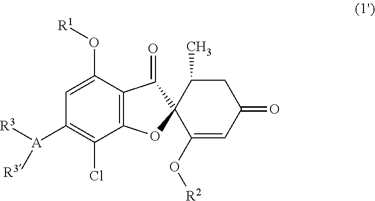

wherein:
R¹ is a C1-C6 alkyl group optionally substituted with the same or different one to two substituents selected from the substituent group X,
a C3-C6 cycloalkyl group optionally substituted with the same or different one to two substituents selected from the substituent group X, or
a 4-7 membered saturated heterocyclic group optionally substituted with the same or different one to two substituents selected from the substituent group X
R² is a C1-C6 alkyl group optionally substituted with the same or different one to two substituents selected from the substituent group X,
a C3-C6 cycloalkyl group optionally substituted with the same or different one to two substituents selected from the substituent group X, or
a 4-7 membered saturated heterocyclic group optionally substituted with the same or different one to two substituents selected from the substituent group X.
A is a 5-membered aromatic heterocyclic ring,
a 6-membered aromatic heterocyclic ring,
an 8-10 membered condensed aromatic heterocyclic ring,
a 5-7 membered unsaturated heterocyclic ring,
a 4-7 membered saturated heterocyclic ring,
a benzene ring, or a single bond, wherein when it is a single bond, one or the other of R³ and R³′ is not present,
R³ and R³′ are each independently a hydrogen atom, a halogen atom, a cyano group, a hydroxy group,
a C1-C6 alkyl group optionally substituted with the same or different one to two substituents selected from the substituent group X,
a C1-C6 alkoxy group optionally substituted with the same or different one to two substituents selected from the substituent group X,
a C2-C6 alkenyl group optionally substituted with the same or different one to two substituents selected from the substituent group X,
a C2-C6 alkynyl group optionally substituted with the same or different one to two substituents selected from the substituent group X,
a C3-C6 cycloalkyl group optionally substituted with the same or different one to two substituents selected from the substituent group X,
an amino group optionally substituted with the same or different one to two substituents selected from the substituent group X,
a C1-C6 alkoxycarbonyl group optionally substituted with the same or different one to two substituents selected from the substituent group X,
a carbamoyl group optionally substituted with the same or different one to two substituents selected from the substituent group X,
a phenyl group optionally substituted with the same or different one to two substituents selected from the substituent group X,
a 5-membered aromatic heterocyclic group optionally substituted with the same or different one to two substituents selected from the substituent group X,
a 6-membered aromatic heterocyclic group optionally substituted with the same or different one to two substituents selected from the substituent group X,
a 5-7 membered unsaturated heterocyclic group optionally substituted with the same or different one to two substituents selected from the substituent group X,
a 4-7 membered saturated heterocyclic group optionally substituted with the same or different one to two substituents selected from the substituent group X,
an 8-10 membered condensed aromatic heterocyclic group optionally substituted with the same or different one to two substituents selected from the substituent group X,
or
R³ and R³′ may form a 5-7 membered unsaturated heterocyclic ring, a 4-7 membered saturated heterocyclic ring, or a C3-C6 cycloalkyl ring as a ring that binds to each other and condenses with A, and the ring is optionally substituted with the same or different one to two substituents selected from the substituent group X,
substituent group X is a halogen atom, a cyano group, a hydroxy group, an oxo group, a C1-C6 alkyl group, a hydroxy C1-C6 alkyl group, a C1-C6 alkoxy C1-C6 alkyl group, a C1-C6 haloalkyl group, a C3-C6 cycloalkyl group, a C3-C6 halocycloalkyl group,
a phenyl group optionally substituted with the same or different one to two substituents selected from the substituent group Y,
a 5-membered aromatic heterocyclic group optionally substituted with the same or different one to two substituents selected from the substituent group Y,
a 6-membered aromatic heterocyclic group optionally substituted with the same or different one to two substituents selected from the substituent group Y,
a 4-7 membered saturated heterocyclic group optionally substituted with the same or different one to two substituents selected from the substituent group Y,
a C1-C6 alkoxy group, a C1-C6 haloalkoxy group, a C3-C6 cycloalkoxy group, a C3-C6 halocycloalkoxy group,
a phenoxy group optionally substituted with the same or different one to two substituents selected from the substituent group Y,
a 5-membered aromatic heterocyclic oxy group optionally substituted with the same or different one to two substituents selected from the substituent group Y,
a 6-membered aromatic heterocyclic oxy group optionally substituted with the same or different one to two substituents selected from the substituent group Y,
a 4-7 membered saturated heterocyclic oxy group optionally substituted with the same or different one to two substituents selected from the substituent group Y,
a C1-C6 alkoxycarbonyl group, a C3-C6 cycloalkoxycarbonyl group, a carboxy group, a C1-C6 alkylcarbonyl group, a C3-C6 cycloalkylcarbonyl group,
a phenylcarbonyl group optionally substituted with the same or different one to two substituents selected from the substituent group Y,
a carbamoyl group, a mono (C1-C6 alkyl)aminocarbonyl group, a di (C1-C6 alkyl) aminocarbonyl group, a mono (C1-C6 alkyl)aminosulfonyl group, a di (C1-C6 alkyl) aminosulfonyl group, an amino group, a mono (C1-C6 alkyl) amino group, a di (C1-C6 alkyl) amino group, a C1-C6 alkoxycarbonylamino group, a mono (C1-C6 alkyl)aminocarbonylamino group, a di (C1-C6 alkyl)aminocarbonylamino group, a C1-C6 alkylcarbonylamino group,
a phenylcarbonylamino group optionally substituted with the same or different one to two substituents selected from the substituent group Y,
a 5-membered aromatic heterocyclic carbonylamino group optionally substituted with the same or different one to two substituents selected from the substituent group Y,
a 6-membered aromatic heterocyclic carbonylamino group optionally substituted with the same or different one to two substituents selected from the substituent group Y, or
a C1-C6 alkylsulfonylamino group, and
Substituent group Y is a C1-C6 alkyl group, a C1-C6 alkoxy group, a halogen atom, or a hydroxy group.

In some embodiments of the compound of Formula 1 or Formula 1′, R¹ is a C1-C6 alkyl group, R² is a C1-C6 alkyl group, A is a 5-membered aromatic heterocyclic ring, and R³ and R³′ are each independently a hydrogen or a C1-C6 alkyl group.

In some embodiments of the compound of Formula 1 or Formula 1′, R′ is a methyl group, an ethyl group, or a hydroxyethyl group.

In some embodiments of the compound of Formula 1 or Formula 1′, R² is a methyl group.

In some embodiments of the compound of Formula 1 or Formula 1′, A is a 5-membered aromatic heterocyclic ring, R³ is a methyl group, an ethyl group, a hydroxy C1-C3 alkyl group, or a methoxy C1-C3 alkyl group, and R³′ is a hydrogen atom.

In some embodiments, the compound of Formula 1 is a compound of a Formula 1″ or a pharmacologically acceptable salt thereof:

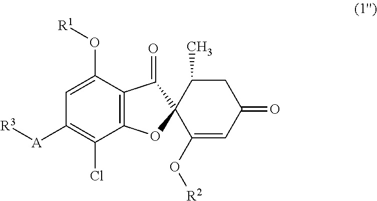

wherein R¹ is a methyl group or an ethyl group;
R² is a methyl group;
A is any ring selected from the following group:

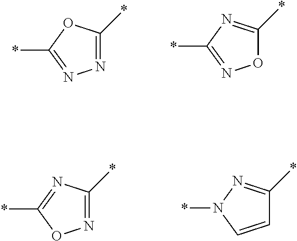 [SMILES: *c1nnc(*)o1 ; *c1noc(*)n1 ; *c1noc(*)n1 ; *c1ccn(*)n1]

• indicates a binding group; and
R³ is a methyl group or an ethyl group.

In some embodiments, the compound of Formula 1′ is any compound selected from the following group:
(2S,5′R)-7-chloro-6-(2-hydroxyethoxy)-3′,4-dimethoxy-5′-methyl-spiro[benzofuran-2,4′-cyclohex-2-ene]-1′,3-dione;
(2S,5′R)-7-chloro-3′,4-dimethoxy-6-(2-methoxyethoxy)-5′-methyl-spiro[benzofuran-2,4′-cyclohex-2-ene]-1′,3-dione;
(2S,5′R)-7-chloro-3′,4-dimethoxy-5′-methyl-6-(1-methylpyrazol-3-yl) spiro[benzofuran-2,4′-cyclohex-2-ene]-1′,3-dione;
(2S,5′R)-7-chloro-6-(1-ethylpyrazol-3-yl)-3′,4-dimethoxy-5′-methyl-spiro[benzofuran-2,4′-cyclohex-2-ene]-1′,3-dione;
(2S,5′R)-7-chloro-3′,4-dimethoxy-5′-methyl-6-(5-methyl-1,3,4-oxadiazol-2-yl) spiro[benzofuran-2,4′-cyclohex-2-ene]-1′,3-dione;
(2S,5′R)-7-chloro-3′,4-dimethoxy-5′-methyl-6-(3-methyl-1,2,4-oxadiazol-5-yl) spiro[benzofuran-2,4′-cyclohex-2-ene]-1′,3-dione;
(2S,5′R)-7-chloro-3′,4-dimethoxy-5′-methyl-6-(5-methyl)-1,2,4-oxadiazol-3-yl) spiro[benzofuran-2,4′-cyclohex-2-ene]-1′,3-dione;
(2S,5′R)-7-chloro-6-[5-(1-hydroxy-1-methyl-ethyl)-1,3,4-oxadiazol-2-yl]-3′,4-dimethoxy-5′-met hyl-spiro[benzofuran-2,4′-cyclohex-2-ene]-1′,3-dione;
(2S,5′R)-7-chloro-6-[5-[(1S)-1-hydroxyethyl]-1,3,4-oxadiazol-2-yl]-3′,4-dimethoxy-5′-methyl-s piro [benzofuran-2,4′-cyclohex-2-ene]-1′,3-dione;
(2S,5′R)-7-chloro-6-[5-[(1R)-1-hydroxyethyl]-1,3,4-oxadiazol-2-yl]-3′,4-dimethoxy-5′-methyl-s piro [benzofuran-2,4′-cyclohex-2-ene]-1′,3-dione;
(2S,5′R)-7-chloro-4-ethoxy-6-[5-(1-hydroxy-1-methyl-ethyl)-1,3,4-oxadiazol-2-yl]-3′-methoxy-5′-methyl-spiro[benzofuran-2,4′-cyclohex-2-ene]-1′,3-dione;
(2S,5′R)-7-chloro-4-ethoxy-6-[5-[(1S)-1-hydroxyethyl]-1,3,4-oxadiazol-2-yl]-3′-methoxy-5′-me thyl-spiro[benzofuran-2,4′-cyclohex-2-ene]-1′,3-dione;
(2S,5′R)-7-chloro-6-[3-(1-hydroxyethyl)-1,2,4-oxadiazol-5-yl]-3′,4-dimethoxy-5′-methyl-spiro[benzofuran-2,4′-cyclohex-2-ene]-1′,3-dione;
(2S,5′R)-7-chloro-4-(2-hydroxyethoxy)-3′-methoxy-5′-methyl-6-(5-methyl-1,3,4-oxadiazol-2-yl) spiro[benzofuran-2,4′-cyclohex-2-ene]-1′,3-dione;
(2S,5′R)-7-chloro-4-(2-hydroxyethoxy)-3′-methoxy-5′-methyl-6-(3-methyl-1,2,4-oxadiazol-5-yl) spiro[benzofuran-2,4′-cyclohex-2-ene]-1′,3-dione;
(2S,5′R)-7-chloro-4-(2-hydroxyethoxy)-3′-methoxy-5′-methyl-6-(5-methyl-1,2,4-oxadiazol-3-yl) spiro[benzofuran-2,4′-cyclohex-2-ene]-1′,3-dione;
(2S,5′R)-7-chloro-6-(5-ethyl-1,3,4-oxadiazol-2-yl)-3′,4-dimethoxy-5′-methyl-spiro[benzofuran-2,4′-cyclohex-2-ene]-1′,3-dione;
(2S,5′R)-7-chloro-3′,4-dimethoxy-5′-methyl-6-(5-tetrahydropyran-4-yl-1,3,4-oxadiazol-2-yl) spiro[benzofuran-2,4′-cyclohex-2-ene]-1′,3-dione;
(2S,5′R)-7-chloro-3′,4-dimethoxy-5′-methyl-6-[5-(1-methyl-4-piperidyl)-1,3,4-oxadiazol-2-yl] spiro[benzofuran-2,4′-cyclohex-2-ene]-1′,3-dione;
(2S,5′R)-7-chloro-6-[5-(4-fluoro-1-methyl-4-piperidyl)-1,3,4-oxadiazol-2-yl]-3′,4-dimethoxy-5′ methyl-spiro[benzofuran-2,4′-cyclohex-2-ene]-1′,3-dione;
(2S,5′R)-7-chloro-3′,4-dimethoxy-6-[5-[(1S)-1-methoxyethyl]-1,3,4-oxadiazol-2-yl]-5′-methyl-s piro [benzofuran-2,4′-cyclohex-2-ene]-1′,3-dione;
(2S,5′R)-7-chloro-4-ethoxy-3′-methoxy-5′-methyl-6-(5-methyl-1,3,4-oxadiazol-2-yl) spiro[benzofuran-2,4′-cyclohex-2-ene]-1′,3-dione;
(2S,5′R)-7-chloro-4-(difluoromethoxy)-3′-methoxy-5′-methyl-6-(5-methyl-1,3,4-oxadiazol-2-yl) spiro[benzofuran-2,4′-cyclohex-2-ene]-1′,3-dione;
(2S,5′R)-7-chloro-3′,4-dimethoxy-6-[3-(1-methoxyethyl)-1,2,4-oxadiazol-5-yl]-5′-methyl-spiro[benzofuran-2,4′-cyclohex-2-ene]-1′,3-dione;
(2S,5′R)-7-chloro-6-[3-(1-hydroxy-1-methyl-ethyl)-1,2,4-oxadiazol-5-yl]-3′,4-dimethoxy-5′-met hyl-spiro[benzofuran-2,4′-cyclohex-2-ene]-1′,3-dione;
(2S,5′R)-7-chloro-3′,4-dimethoxy-5′-methyl-6-(1H-pyrazol-5-yl) spiro[benzofuran-2,4′-cyclohex-2-ene]-1′,3-dione;
(2S,5′R)-7-chloro-3′,4-dimethoxy-6-[1-(2-methoxyethyl) pyrazol-3-yl]-5′-methyl-spiro[benzofuran-2,4′-cyclohex-2-ene]-1′,3-dione;
(2S,5′R)-7-chloro-6-(1,8-dioxa-2-azaspiro[4.5]dec-2-en-3-yl)-3′,4-dimethoxy-5′-methyl-spiro[benzofuran-2,4′-cyclohex-2-ene]-1′,3-dione;
(2S,5′R)-7-chloro-3′,4-dimethoxy-5′-methyl-6-(8-methyl-1-oxa-2,8-diazaspiro[4.5]dec-2-en-3-yl) spiro[benzofuran-2,4′-cyclohex-2-ene]-1′,3-dione;
(2S,5′R)-7-chloro-3′,4-dimethoxy-6-(2-methoxypyrimidin-5-yl)-5′-methyl-spiro[benzofuran-2,4′-cyclohex-2-ene]-1′,3-dione;
(2S,5′R)-7-chloro-3′,4-dimethoxy-6-(6-methoxy-3-pyridyl)-5′-methyl-spiro[benzofuran-2,4′-cyclohex-2-ene]-1′,3-dione;
(2S,5′R)-7-chloro-3′,4-dimethoxy-5′-methyl-6-(3-pyridyl) spiro[benzofuran-2,4′-cyclohex-2-ene]-1′,3-dione; or
(2S,5′R)-7-chloro-3′,4,6-trimethoxy-5′-methyl-spiro[benzofuran-2,4′-cyclohex-2-ene]-1′,3-dione.

In one embodiment, the compound is (2S,5′R)-7-chloro-6-(1-ethylpyrazol-3-yl)-3′,4-dimethoxy-5′-methyl-spiro[benzofuran-2,4′-cyclohex-2-ene]-1′,3-dione or a pharmacologically acceptable salt thereof.

In one embodiment, the compound is (2S,5′R)-7-chloro-3′,4-dimethoxy-5′-methyl-6-(5-methyl-1,3,4-oxadiazol-2-yl) spiro[benzofuran-2,4′-cyclohex-2-ene]-1′,3-dione or a pharmacologically acceptable salt thereof.

In one embodiment, the compound is (2S,5′R)-7-chloro-6-(5-ethyl-1,3,4-oxadiazol-2-yl)-3′,4-dimethoxy-5′-methyl-spiro[benzofuran-2,4′-cyclohex-2-ene]-1′,3-dione or a pharmacologically acceptable salt thereof.

In one embodiment, the compound is (2S,5′R)-7-chloro-6-[3-(1-hydroxy-1-methyl-ethyl)-1,2,4-oxadiazol-5-yl]-3′,4-dimethoxy-5′-methyl-spiro[benzofuran-2,4′-cyclohex-2-ene]-1′,3-dione or a pharmacologically acceptable salt thereof.

In one embodiment, the compound is (2S,5′R)-7-chloro-4-ethoxy-3′-methoxy-5′-methyl-6-(5-methyl-1,3,4-oxadiazol-2-yl) spiro[benzofuran-2,4′-cyclohex-2-ene]-1′,3-dione or a pharmacologically acceptable salt thereof.

In certain embodiments, the 5-membered aromatic heterocyclic ring for A is the same as described above, but more preferably, it represents the following 5-membered ring. (It should be noted that in this case, R³′ is not present.)

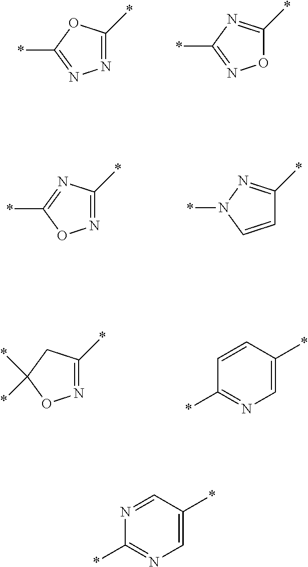

wherein * indicates a binding group.

In the present specification, the “5-membered aromatic heterocyclic ring” is a monocyclic 5-membered aromatic heterocyclic ring containing one to four atoms selected from the group consisting of a nitrogen atom, an oxygen atom, and a sulfur atom. For example, rings such as those shown below are included.

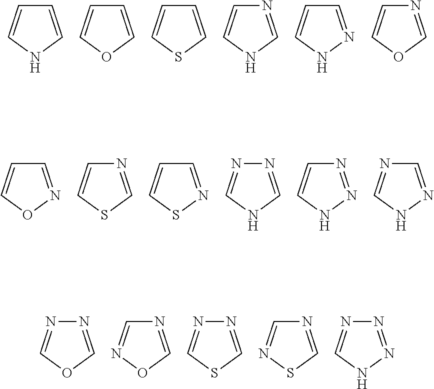

In the present specification, the “6-membered aromatic heterocyclic ring” is a monocyclic 6-membered aromatic heterocyclic ring containing one to four atoms selected from the group consisting of a nitrogen atom, an oxygen atom, and a sulfur atom. For example, rings such as those shown below are included.

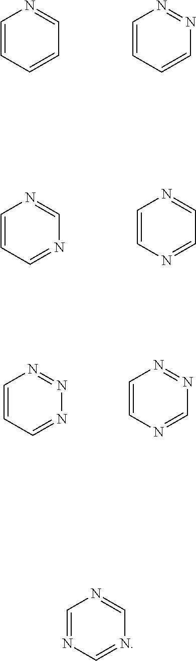

In the present specification, the “8-10 membered condensed aromatic heterocyclic ring” is an 8-10 membered condensed aromatic heterocyclic ring containing one to four atoms selected from the group consisting of a nitrogen atom, an oxygen atom, and a sulfur atom. For example, rings such as those shown below are included.

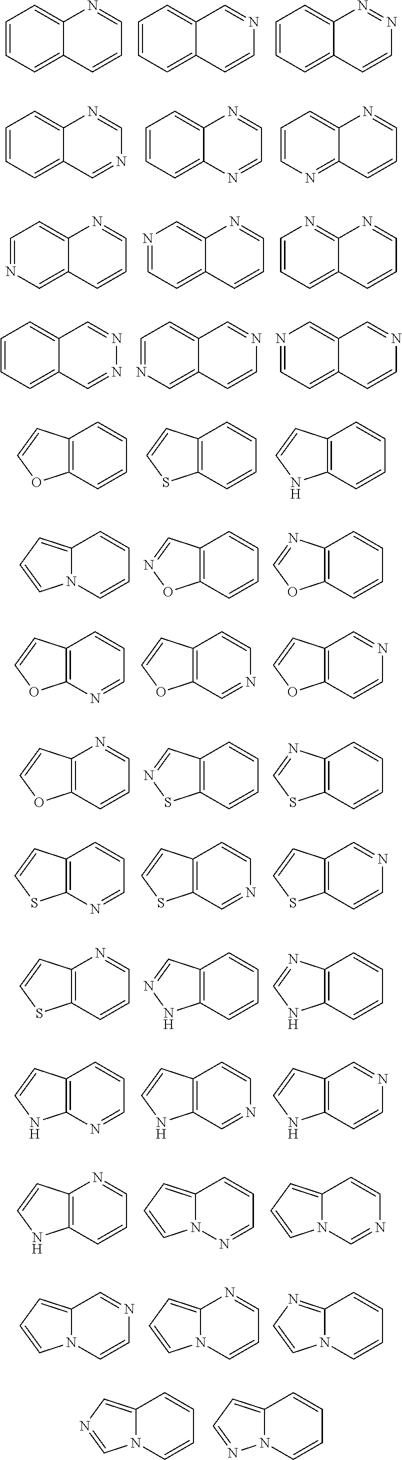

In the present specification, the “5-7 membered unsaturated heterocyclic ring” is a ring in which a monocyclic 5-7 membered saturated heterocyclic ring is partially oxidized or a ring in which an aromatic heterocyclic ring is partially reduced containing one to four atoms selected from the group consisting of a nitrogen atom, an oxygen atom, and a sulfur atom. For example, rings such as those shown below are included.

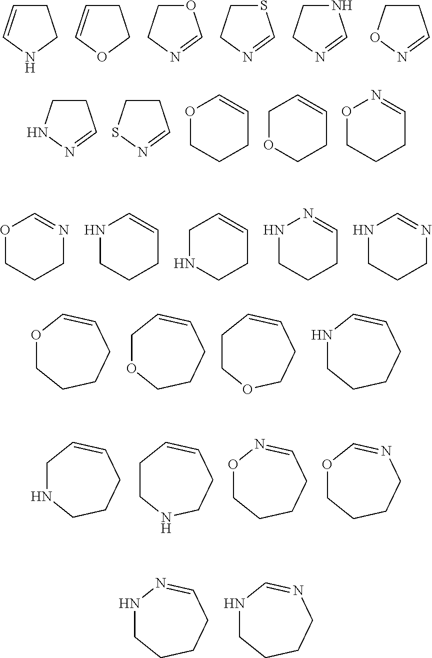

In the present specification, the “4-7 membered saturated heterocyclic ring” is a monocyclic 4-7 membered saturated heterocyclic ring containing one to four atoms selected from the group consisting of a nitrogen atom, an oxygen atom, and a sulfur atom. For example, rings such as those shown below are included.

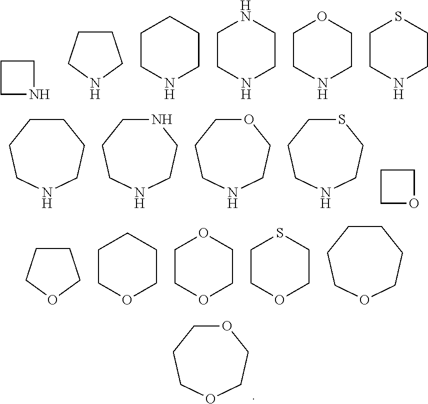

The “halogen atom” in the present specification is a fluorine atom, a chlorine atom, a bromine atom, or an iodine atom, and is preferably a fluorine atom or a chlorine atom.

The “C1-C6 alkyl group” in the present specification is a linear or branched alkyl group having one to six carbon atoms. Examples thereof include a methyl group, an ethyl group, a 1-propyl group, an isopropyl group, a 1-butyl group, a 2-butyl group, a 2-methyl-1-propyl group, a 2-methyl-2-propyl group, a 1-pentyl group, a 2-pentyl group, a 3-pentyl group, a 2-methyl-2-butyl group, a 3-methyl-2-butyl group, a 1-hexyl group, a 2-hexyl group, a 3-hexyl group, a 2-methyl-1-pentyl group, a 3-methyl-1-pentyl group, a 2-ethyl-1-butyl group, a 2,2-dimethyl-1-butyl group, and a 2,3-dimethyl-1-butyl group, and it is preferably a methyl group or an ethyl group.

The “C2-C6 alkenyl group” in the present specification is a linear or branched alkenyl group having two to six carbon atoms, and it may have one or two or more carbon-carbon double bonds. For example, it is a vinyl group, a 2-propenyl (allyl) group, a 2-butenyl group, a 2-pentenyl group, a 3-methyl-2-butenyl group, a 2-hexenyl group, or a 3-methyl-2-pentenyl group, and preferably, it is a vinyl group or an allyl group.

The “C2-C6 alkynyl group” in the present specification is a linear or branched alkynyl group having two to six carbon atoms, and it may have one or two or more carbon-carbon triple bonds. For example, it is an ethynyl group, a 1-propynyl group, a 2-propynyl group, a 1-butynyl group, a 2-butynyl group, a 1-pentynyl group, a 2-pentynyl group, or 1-hexynyl group, and it is preferably an ethynyl group or a 1-propynyl group.

The “C1-C6 alkoxy group” in the present specification is a group in which an oxygen atom is bonded to a C1-C6 alkyl group. Examples thereof include a methoxy group, an ethoxy group, a 1-propoxy group, a 2-propoxy group, a 1-butoxy group, a 2-butoxy group, a 2-methyl-1-propoxy group, a 2-methyl-2-propoxy group, a 1-pentyloxy group, a 2-pentyloxy group, a 3-pentyloxy group, a 2-methyl-2-butoxy group, a 3-methyl-2-butoxy group, a 1-hexyloxy group, a 2-hexyloxy group, a 3-hexyloxy group, a 2-methyl-1-pentyloxy group, and a 3-methyl-1-pentyloxy group. Preferably, it is a methoxy group, an ethoxy group, a 1-propoxy group, or a 2-propoxy group.

The “C3-C6 cycloalkyl group” in the present specification is a cyclic alkyl group having three to six carbon atoms, and it is preferably a cyclopropyl group, a cyclobutyl group, a cyclopentyl group, or a cyclohexyl group.

The “hydroxy C1-C6 alkyl group” in the present specification is a group in which a hydroxyl group is bonded to a C1-C6 alkyl group. For example, it is a hydroxymethyl group or a hydroxyethyl group.

The “C1-C6 alkoxy C1-C6 alkyl group” in the present specification is a group in which a C1-C6 alkoxy is bonded to a C1-C6 alkyl group. Examples thereof include a methoxymethyl group, a methoxyethyl group, an ethoxymethyl group, and an ethoxyethyl group.

The “C1-C6 haloalkyl group” in the present specification is a group in which a halogen atom is bonded to a C1-C6 alkyl group. Examples thereof include a fluoromethyl group, a difluoromethyl group, a dichloromethyl group, a dibromomethyl group, a trifluoromethyl group, a trichloromethyl group, a 2-fluoroethyl group, a 2-bromoethyl group, a 2-chloroethyl group, a 2-iodoethyl group, a 2,2-difluoroethyl group, a 2,2,2-trifluoroethyl group, a trichloroethyl group, a pentafluoroethyl group, a 3-fluoropropyl group, a 3-chloropropyl group, and a 4-fluorobutyl group. It is preferably a trifluoromethyl group.

The “C3-C6 halocycloalkyl group” in the present specification is a group in which a halogen atom is bonded to a C3-C6 cycloalkyl group, and examples thereof include a fluorocyclopropyl group, a fluorocyclobutyl group, a fluorocyclopentyl group, and a fluorocyclohexyl group.

The “C1-C6 haloalkoxy group” in the present specification is a group in which a halogen atom is bonded to a C1-C6 alkoxy group, and examples thereof include a fluoromethoxy group, a difluoromethoxy group, a dichloromethoxy group, a dibromomethoxy group, a trifluoromethoxy group, a trichloromethoxy group, a 2-fluoroethoxy group, a 2-bromoethoxy group, a 2-chloroethoxy group, a 2-iodoethoxy group, a 2,2-difluoroethoxy group, a 2,2,2-trifluoroethoxy group, a trichloroethoxy group, a pentafluoroethoxy group, a 3-fluoropropoxy group, a 3-chloropropoxy group, and a 4-fluorobutoxy group. It is preferably a trifluoromethoxy group.

The “C3-C6 cycloalkoxy group” in the present specification is a group in which a C3-C6 cycloalkyl group is bonded to an oxygen atom, and it is preferably a cyclopropyloxy group, a cyclobutyloxy group, a cyclopentyloxy group, or a cyclohexyloxy group.

The “C3-C6 halocycloalkoxy group” in the present specification is a group in which a C3-C6 halocycloalkyl group is bonded to an oxygen atom, and examples thereof include a fluorocyclopropoxy group, a fluorocyclobutoxy group, a fluorocyclopentyloxy group, and a fluorocyclohexyloxy group.

The “5-membered aromatic heterocyclic oxy group” in the present specification is a group in which a 5-membered aromatic heterocyclic ring is bonded to an oxygen atom.

The “6-membered aromatic heterocyclic oxy group” in the present specification is a group in which a 6-membered aromatic heterocyclic ring is bonded to an oxygen atom.

The “4-7 membered saturated heterocyclic oxy group” in the present specification is a group in which a 4-7 membered saturated heterocyclic ring is bonded to an oxygen atom.

The “C1-C6 alkoxycarbonyl group” in the present specification is a group in which a C1-C6 alkoxy group is bonded to a carbonyl group, and examples thereof include a methoxycarbonyl group, an ethoxycarbonyl group, and a propoxycarbonyl group.

The “C3-C6 cycloalkoxycarbonyl group” in the present specification is a group in which a C3-C6 cycloalkoxy group is bonded to a carbonyl group, and it is preferably a cyclopropyloxycarbonyl group, a cyclobutyloxycarbonyl group, a cyclopentyloxycarbonyl group, or a cyclohexyloxycarbonyl group.

The “C1-C6 alkyl carbonyl group” in the present specification is a group in which a C1-C6 alkyl group is bonded to a carbonyl group, and examples thereof include a methyl carbonyl group, an ethyl carbonyl group, or a propyl carbonyl group.

The “mono (C1-C6 alkyl)aminocarbonyl group” in the present specification is a group in which one C1-C6 alkyl group is bonded to the amino group of an aminocarbonyl group, and it is preferably a methylaminocarbonyl group, an ethylaminocarbonyl group, or a propylaminocarbonyl group.

The “di (C1-C6 alkyl)aminocarbonyl group” in the present specification is a group in which two C1-C6 alkyl groups are bonded to the amino group of an aminocarbonyl group, and it is preferably a dimethylaminocarbonyl group, a diethylaminocarbonyl group, or a dipropylaminocarbonyl group.

The “mono (C1-C6 alkyl)aminosulfonyl group” in the present specification is a group in which one C1-C6 alkyl group is bonded to the amino group of an aminosulfonyl group, and it is preferably a methylaminosulfonyl group, an ethylaminosulfonyl group, or a propylaminosulfonyl group

The “di (C1-C6 alkyl)aminosulfonyl group” in the present specification is a group in which two C1-C6 alkyl groups are bonded to the amino group of the aminosulfonyl group, and it is preferably a dimethylaminosulfonyl group, a diethylaminosulfonyl group, or a dipropylaminosulfonyl group.

The “mono (C1-C6 alkyl) amino group” in the present specification is a group in which one C1-C6 alkyl group is bonded to an amino group, and it is preferably a methylamino group, an ethylamino group, or a propylamino group.

The “di (C1-C6 alkyl) amino group” in the present specification is a group in which two C1-C6 alkyl groups are bonded to an amino group, and it is preferably a dimethylamino group, a diethylamino group, or a dipropyl amino group.

The “C1-C6 alkoxycarbonylamino group” in the present specification is a group in which a C1-C6 alkoxycarbonyl group is bonded to an amino group, and for example, it is a methoxycarbonylamino group, an ethoxycarbonylamino group, or a propoxycarbonylamino group.

The “mono (C1-C6 alkyl)aminocarbonylamino group” in the present specification is a group in which a mono (C1-C6 alkyl)aminocarbonyl group is bonded to an amino group, and it is preferably a methylaminocarbonylamino group, an ethylaminocarbonylamino group, or a propylaminocarbonylamino group.

The “di (C1-C6 alkyl)aminocarbonylamino group” in the present specification is a group in which a di (C1-C6 alkyl)aminocarbonyl group is bonded to an amino group, and it is preferably a dimethylaminocarbonylamino group, a diethylaminocarbonylamino group, or a dipropylaminocarbonylamino group.

The “5-membered aromatic heterocyclic carbonylamino group” in the present specification is a group in which a 5-membered aromatic heterocyclic carbonyl group is bonded to an amino group.

The “6-membered aromatic heterocyclic carbonylamino group” in the present specification is a group in which a 6-membered aromatic heterocyclic carbonyl group is bonded to an amino group.

The “C1-C6 alkylsulfonylamino group” in the present specification is a group in which a C1-C6 alkyl group is bonded to the sulfonyl group of a sulfonylamino group, and it is preferably a methylsulfonylamino group, an ethylsulfonylamino group, or a propylsulfonylamino group.

The “pharmaceutically acceptable salt” indicates a salt that can be used as a pharmaceutical. When the compound has an acidic group or a basic group it can be converted to a basic salt or an acidic salt by reacting with a base or an acid to form a salt thereof.

The pharmaceutically acceptable “basic salt” of the compound preferably includes an alkali metal salt such as a sodium salt, a potassium salt, and a lithium salt; an alkaline earth metal salt such as a magnesium salt and a calcium salt; organic base salts such as an N-methyl morpholine salt, a triethylamine salt, a tributylamine salt, a diisopropylethylamine salt, a dicyclohexylamine salt, an N-methylpiperidine salt, a pyridine salt, a 4-pyrrolidinopyridine salt, and a picoline salt; and an amino acid salt such as glycine salt, a lysine salt, an arginine salt, an ornithine salt, a glutamate, and an aspartate, and it is preferably an alkali metal salt.

The pharmaceutically acceptable “acidic salt” of the compound preferably includes an inorganic acid salt such as a hydrohalide such as a hydrofluoride, a hydrochloride, a hydrobromide, and a hydroiodide, a nitrate, a perchlorate, a sulfate, and a phosphate; an organic salt such as a lower alkanesulfonate such as methanesulfonate, trifluoromethanesulfonate, and ethanesulfonate, an aryl sulfonate such as a benzenesulfonates, and a p-toluene sulfonate, an acetate, a malate, a fumarate, a succinate, a citrate, an ascorbate, a tartrate, an oxalate, a maleate, and the like; and an amino acid salt such as glycine salt, a lysine salt, an arginine salt, an ornithine salt, a glutamate, and an aspartate, and it is most preferably a hydrohalide (in particular, a hydrochloride).

The compound of the present invention or the pharmaceutically acceptable salt thereof may absorb moisture, adhere to adsorbed water, or become a hydrate by leaving in the air or recrystallization. The present invention also encompasses compounds of such various hydrates, solvates, and crystalline polymorphs.

The compounds of the present invention, their pharmaceutically acceptable salts or solvates thereof, depending on the type and combination of substituents, may have various isomers such as geometric isomers such as a cis isomer and a trans isomer, tautomers, or optical isomers such as a d isomer and an 1 isomer, while the compounds include those all isomers, stereoisomers, and mixtures of these isomers and stereoisomers in any ratio unless otherwise specified. Mixtures of these isomers may be resolved by known resolution means.

The compounds of the present invention also include labels, that is, a compound in which one or more atoms of the compounds are substituted with an isotope (for example, 2H, 3H, 13C, 14C, 35S, and the like).

In addition, the present invention also encompasses a prodrug. The prodrug is a compound having a group which can be converted to an amino group, a hydroxyl group, a carboxyl group, or the like of the compound by hydrolysis or under physiological conditions, and as a group forming such a prodrug, it is a group described in Prog. Med., Vol. 5, pp. 2157 to 2161 (1985) or the like. As the prodrug, more specifically, when an amino group is present in the compound, a compound in which the amino group is acylated, alkylated, or phosphorylated (for example, it is a compound in which the amino group is eicosanoylated, alanylated, pentylaminocarbonylated, (5-methyl-2-oxo-1,3-dioxolen-4-yl) methoxycarbonylated, tetrahydrofuranylated, pyrrolidinyl methylated, pivaloyloxymethylatied, or tert-butylated, or the like) and the like are included, and when a hydroxyl group is present in the compound, a compound in which the hydroxyl group is acylated, alkylated, phosphorylated, or borated (for example, it is a compound in which the hydroxyl group is acetylated, palmitoylated, propanoylated, pivaloylated, succinylated, fumarylated, alanylated, or dimethylaminomethyl carbonylated, or the like.) and the like are included. In addition, when a carboxy group is present in the compound, a compound in which the carboxy group is esterified or amidated (for example, it is a compound in which the carboxy group is ethyl esterified, phenyl esterified, carboxymethyl esterified, dimethylaminomethyl esterified, pivaloyloxymethyl esterified, ethoxycarbonyloxyethyl esterified, amidated, or methylamidated, or the like.), and the like are included.

The compounds of the present invention may be produced by synthetic methods known in the art and as described in WO 2017/170623 and WO 2019/065928, incorporated by reference herein in their entirety.

Administration of the compounds of the present invention may be carried out by any form of oral administration by a tablet, a pill, a capsule, a granule, a powder, a solution, or the like, or by any form of parenteral administration by an injection for intra-articular, intravenous, intramuscular, or the like, a suppository, an eye drop, an eye ointment, a transdermal solution, an ointment, a transdermal patch, a transmucosal solution, a transmucosal patch, an inhalant, or the like.

As a solid composition for oral administration, a tablet, a powder, a granule, and the like are used. Such a solid composition is composed of one or more active ingredients and at least one inert excipient such as lactose, mannitol, glucose, hydroxypropyl cellulose, microcrystalline cellulose, starch, polyvinyl pyrrolidone, magnesium metasilicate aluminate, and/or the like. The solid composition may contain, according to a conventional method, one or more of an inert additive such as a lubricant such as magnesium stearate, a disintegrant such as sodium carboxymethyl starch, a stabilizer, and a solubilizer. The tablet or pill may be coated with a sugar coating or a film of a substance soluble in the stomach or intestine, if necessary.

As a liquid composition for oral administration, a pharmaceutically acceptable emulsion, solution, suspension, syrup, elixir, or the like is used. To such a liquid composition, it is possible to add a generally used inert diluent such as purified water or ethanol. The liquid composition may contain, in addition to an inert diluent, one or more of a solubilizer, an adjuvant such as a wetting agent, a sweetening agent, a flavoring agent, a fragrance, and a preservative.

As an injection for parenteral administration, a sterile aqueous or non-aqueous solution, a suspension or an emulsion, and the like are used. The aqueous solvent includes, for example, distilled water for injection, physiological saline, and the like. The non-aqueous solvents include, for example, propylene glycol, polyethylene glycol, and vegetable oil such as olive oil, alcohols such as ethanol, Polysorbate 80, and the like. Such an injection composition may further contain a one or more of a tonicity agent, a preservative, a wetting agent, an emulsion, a dispersing agent, a stabilizer, or a solubilizer. These injection compositions can be sterilized by, for example, filtration through a bacteria retention filter, application of a bactericide, or irradiation. In addition, these injection compositions may be used by producing a sterile solid composition and dissolved or suspended in sterile water or a sterile solvent for injection prior to use.

As an external preparation, an ointment, a plaster, a cream, a jelly, a cataplasm, a spray, a lotion, an eye drop, an eye ointment, and the like are used. These external preparations include generally used ointment bases, lotion bases, aqueous or non-aqueous solutions, suspensions, emulsions, and the like. For example, as an ointment or lotion base, polyethylene glycol, propylene glycol, white petrolatum, bleached beeswax, polyoxyethylene hydrogenated castor oil, glycerin monostearate, stearyl alcohol, cetyl alcohol, lauromacrogol, sorbitan sesquioleate, and the like are used.

A transmucosal agent such as an inhalant and a transnasal agent are used in solid, liquid, or semisolid form, and it may be produced according to a conventionally known method. For example, a known excipient, and furthermore, one or more of a pH adjuster, a preservative, a surfactant, a lubricant, a stabilizer, a thickener, and the like may be added as appropriate. With these transmucosal agents, devices appropriate for inhalation or insufflation may be used as the method of administration. For example, the compound may be administered alone or as a powder of a formulated mixture, or as a solution or suspension in combination with a pharmaceutically acceptable carrier, using known devices and nebulizers, such as metered dose inhalation devices. A dry powder inhaler or the like may be for single or multiple administration, and a dry powder or powder containing capsule may be also used. Alternatively, an appropriate ejector may be used. For example, it may be in the form of a pressurized aerosol spray or the like using a suitable gas such as chlorofluoroalkane, hydrofluoroalkane, or carbon dioxide.

In the case of normal oral administration, the appropriate daily dose is about 0.001 to 100 mg/kg, preferably 0.1 to 30 mg/kg, and more preferably 0.1 to 10 mg/kg of body weight. This is administered in one dose or separated into two or more doses. In some embodiments, the oral dose is about 1 mg to about 100 mg per day, e.g., about 5 mg to about 50 mg per day, e.g., about 10 mg to about 30 mg per day. When administered intravenously, the appropriate daily dose is about 0.0001 to 10 mg/kg of body weight, which is administered once or separated into several times a day. In addition, as a transmucosal agent, about 0.001 to 100 mg/kg of body weight is administered once or separated into several times a day. The dose is appropriately determined depending on the individual case in consideration of symptoms, age, sex, and the like.

In the methods of the present invention, the compound may be administered in combination with various therapeutic agents or preventive agents for diseases that are considered to exhibit the efficacy thereof. The combination may be administered simultaneously, separately, concurrently, and continuously or at desired time intervals. The co-administered agents may be blended or formulated separately. The therapeutic agent may be, for example, one that treats depression or one that treats an underlying disorder that is causing depression.

The methods of the present invention find use in both veterinary and medical applications. Suitable subjects include avians, reptiles, amphibians, fish, and mammals. The term “mammal” as used herein includes, but is not limited to, humans, primates, non-human primates (e.g., monkeys and baboons), cattle, sheep, goats, pigs, horses, cats, dogs, rabbits, rodents (e.g., rats, mice, hamsters, and the like), etc. Human subjects include neonates, infants, juveniles, and adults. Optionally, the subject is “in need of” the methods of the present invention, e.g., because the subject has or is believed at risk for depression or that would benefit from the delivery of a compound as described herein. As a further option, the subject can be a laboratory animal and/or an animal model of disease. Preferably, the subject is a human.

Having described the present invention, the same will be explained in greater detail in the following examples, which are included herein for illustration purposes only, and which are not intended to be limiting to the invention.

### Example 1

### Treatment of Major Depressive Disorder

A Phase 2, multicenter, double-blind, randomized, placebo-controlled study of the safety and efficacy of SP-624 in the treatment of adult subjects with major depressive disorder (MDD) as defined by the Diagnostic and Statistical Manual of Mental Disorders, Fifth Edition (DSM-5) was carried out. Following the successful completion of a Screening Phase, subjects were randomized to one of two treatment groups in a 1:1 ratio and received Compound 1 (SP-624) or placebo over a treatment period of four weeks (Treatment Group #1: SP-624 20 mg/day; Treatment Group #2: Placebo).

### Compound 1 (SP-624; DS-7830a; (2S,6′R)-7-Chloro-2′,4-dimethoxy-6′-methyl-6-(5-methyl-1,3,4-oxadiazol-2-yl)-3H-spiro[1-benzofuran-2,1′-cyclohex [2]ene]-3,4′-dione)

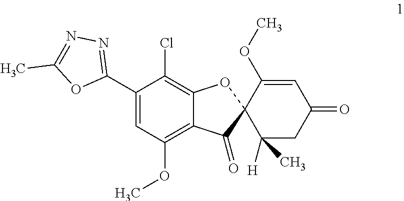 [SMILES: COC1=CC(=O)C[C@@H](C)[C@]12Oc1c(Cl)c(-c3nnc(C)o3)cc(OC)c1C2=O]

Prior to initiating the 4-week treatment period, subjects completed a Screening/Baseline period of up to 28 days, during which time all screening assessments were performed, and any current depression medications discontinued. All screening assessments were completed before discontinuing any current depression medications. Subjects returned for a Baseline Visit to complete efficacy and safety assessments. During the treatment period, subjects returned to the investigative site to complete efficacy and safety assessments at the end of Weeks 1, 2, 3, and 4. After the final dose of study drug, subjects completed a follow-up period of two weeks and returned at the end of Weeks 5 and 6. Subjects may be treated with anti-depressant medications according to physician recommendations after completion of the Week 5 Visit.

Each dose of Compound 1 was supplied as two capsules, each containing 10 mg of active pharmaceutical ingredient (API). Matching placebo capsules identical in shape and color to the active capsules were used.

The primary efficacy endpoint was Change from Baseline to Week 4 in Montgomery-Asberg Depression Rating Scale (MADRS) total score.

Secondary efficacy endpoints included:
Change from Baseline to Weeks 1, 2, and 3 in MADRS total score,
Change from Baseline to Weeks 1, 2, 3, and 4 in Clinical Global Impression-Severity (CGI-S) total score,
Change from Baseline to Week 5 and change from Week 4 to Week 5 in MADRS total score and CGI-S total score,
Change from Baseline to Week 2 and Week 4 in the 17-item Hamilton Depression Rating Scale (HAM D-17) total score,
Change from Baseline to Week 2 and Week 4 in the Sheehan Disability Scale (SDS),
Change from Baseline to Week 2 and Week 4 in the Quick Inventory of Depressive Symptomology-Self Report (QIDS-SR), and
Change from Baseline to Week 2 and Week 4 in the Quality of Life Enjoyment and Satisfaction Questionnaire-Short Form (Q-LES-Q-SF).

The demographics of the study population is shown in Table 1.

TABLE 1
Demographics, Safety Population

| | SP-624 | | Placebo | | Overall | |
|---|---|---|---|---|---|---|
| Summary | (N = 161) | | (N = 156) | | (N = 317) | |
| Age (Years) | | | | | | |
| n | 161 | | 156 | | 317 | |
| Mean (SD) | 41.5 | (13.95) | 43.9 | (14.11) | 42.7 | (14.06) |
| Median | 42.0 | | 44.0 | | 43.0 | |
| Min-Max | 18.0-65.0 | | 18.0-65.0 | | 18.0-65.0 | |
| Gender, n (%) | | | | | | |
| Female | 101 | (62.7%) | 110 | (70.5%) | 211 | (66.6%) |
| Male | 60 | (37.3%) | 46 | (29.5%) | 106 | (33.4%) |
| Race | | | | | | |
| American Indian or | 2 | (1.2%) | 2 | (1.3%) | 4 | (1.3%) |
| Alaska native | | | | | | |
| Asian | 8 | (5.0%) | 12 | (7.7%) | 20 | (6.3%) |
| Black or African | 41 | (25.5%) | 34 | (21.8%) | 75 | (23.7%) |
| American | | | | | | |
| Native Hawaiian or | 2 | (1.2%) | 2 | (1.3%) | 4 | (1.3%) |
| other Pacific Islander | | | | | | |
| White | 113 | (70.2%) | 111 | (71.2%) | 224 | (70.7%) |
| Ethnicity, n (%) | | | | | | |
| Hispanic or Latino | 16 | (9.9%) | 21 | (13.5%) | 37 | (11.7%) |
| Not Hispanic or Latino | 142 | (88.2%) | 135 | (86.5%) | 277 | (87.4%) |
| Nor reported | 1 | (0.6%) | 0 | (0.0%) | 1 | (0.3%) |
| Unknown | 2 | (1.2%) | 0 | (0.0%) | 2 | (0.6%) |

The study results surprisingly showed that Compound 1 was therapeutically effective in females, lowering the MADRS score by an average of 3.9 points, while having no effect on males (Tables 2-5). The results are remarkable, particularly in view of the demonstrated preclinical efficacy of Compound 1, which was carried out in male mice and rats.

TABLE 2
Primary Efficacy Analysis Female Subjects Change from Baseline MADRS Score at Week 4 Using
Mixed Models for Repeated Measures (MMRM), modified intent to treat (mITT) Population

| | | | Change from Baseline | | | |
|---|---|---|---|---|---|---|
| | SP-624 | Placebo | SP-624 | Placebo | LSM Difference | |
| | (N = 98) | (N = 107) | (N = 98) | (N = 107) | (95% CI) | P-value |
| MADRS Total | | | | | | |
| at Baseline | | | | | | |
| n | 98 | 107 | | | | |
| Mean (SD) | 33.4 (4.59) | 33.6 (3.73) | | | | |
| Median | 33 | 33 | | | | |
| Min, Max | 27, 45 | 27, 44 | | | | |
| MADRS Total at | | | | | | |
| Week 4 | | | | | | |
| n | 84 | 86 | 84 | 86 | −3.9 (−6.8, −1.0) | 0.008 |
| Mean (SD) | 19.8 (10.75) | 24.0 (10.06) | −13.5 (10.40) | −9.4 (9.96) | | |
| Median | 21 | 27 | −13 | −6 | | |
| Min, Max | 0, 39 | 0, 39 | −41, 2 | −39, 5 | | |
| LSM | | | −13.4 (1.05) | −9.4 (1.02) | | |
| Estimate (SE) | | | | | | |

TABLE 3
Female Change from Baseline to Pre-Week 4 Timepoints
in MADRS Score Using MMRM, mITT Population

| | | | Change from Baseline | | | |
|---|---|---|---|---|---|---|
| | SP-624 | Placebo | SP-624 | Placebo | LSM Difference¹ | |
| | (N = 98) | (N = 107) | (N = 98) | (N = 107) | (95% CI) | P-value¹ |
| MADRS Total at | | | | | | |
| Baseline | | | | | | |
| n | 98 | 107 | | | | |
| Mean (SD) | 33.4 (4.59) | 33.6 (3.73) | | | | |
| Median | 33 | 33 | | | | |
| Min, Max | 27, 45 | 27, 44 | | | | |
| MADRS Total | | | | | | |
| at Week 1 | | | | | | |
| n | 95 | 105 | 95 | 105 | −0.9 (−2.8, 1.1) | 0.389 |
| Mean (SD) | 26.7 (8.10) | 27.8 (7.58) | −6.6 (6.19) | −5.8 (7.63) | | |
| Median | 27 | 30 | −6 | −3 | | |
| Min, Max | 7, 45 | 1, 42 | −24, 5 | −38, 4 | | |
| LSM Estimate | | | −6.6 (0.71) | −5.8 (0.68) | | |
| (SE) | | | | | | |
| MADRS Total | | | | | | |
| at Week 2 | | | | | | |
| n | 86 | 92 | 86 | 92 | −1.9 (−4.3, 0.5) | 0.119 |
| Mean (SD) | 23.1 (8.76) | 25.5 (8.88) | −10.1 (8.20) | −7.8 (8.68) | | |
| Median | 25 | 28 | −8 | −5 | | |
| Min, Max | 3, 42 | 1, 41 | −35, 2 | −38, 6 | | |
| LSM Estimate | | | −9.7 (0.87) | −7.8 (0.84) | | |
| (SE) | | | | | | |
| MADRS Total | | | | | | |
| at Week 3 | | | | | | |
| n | 89 | 90 | 89 | 90 | −3.5 (−6.2, −0.8) | 0.011 |
| Mean (SD) | 20.8 (10.11) | 24.0 (9.10) | −12.5 (9.74) | −9.4 (9.20) | | |
| Median | 21 | 27 | −11 | −6 | | |
| Min, Max | 0, 41 | 2, 39 | −42, 4 | −32, 8 | | |
| LSM Estimate | | | −12.4 (0.98) | −8.9 (0.95) | | |
| (SE) | | | | | | |

TABLE 4
Primary Efficacy Analysis Male Subjects Change from Baseline
MADRS Score at Week 4 Using MMRM, mITT Population

| | | | Change from Baseline | | | |
|---|---|---|---|---|---|---|
| | SP-624 | Placebo | SP-624 | Placebo | LSM Difference¹ | |
| | (N = 59) | (N = 46) | (N = 59) | (N = 46) | (95% CI) | P-value¹ |
| MADRS Total | | | | | | |
| at Baseline | | | | | | |
| n | 59 | 46 | | | | |
| Mean (SD) | 32.4 (3.74) | 33.0 (4.39) | | | | |
| Median | 32 | 33 | | | | |
| Min, Max | 27, 42 | 27, 43 | | | | |
| MADRS Total | | | | | | |
| at Week 4 | | | | | | |
| n | 48 | 39 | 48 | 39 | 2.7 (−0.8, 6.3) | 0.134 |
| Mean (SD) | 20.8 (9.61) | 18.2 (9.03) | −11.4 (9.44) | −14.8 (9.72) | | |
| Median | 21 | 18 | −10 | −13 | | |
| Min, Max | 0, 41 | 2, 36 | −37, 1 | −36, 2 | | |
| LSM | | | −11.3 (1.21) | −14.0 (1.35) | | |
| Estimate (SE) | | | | | | |

TABLE 5
Male Change from Baseline to Pre-Week 4 Timepoints
in MADRS Score Using MMRM, mITT Population

| | | | Change from Baseline | | | |
|---|---|---|---|---|---|---|
| | SP-624 | Placebo | SP-624 | Placebo | LSM Difference¹ | |
| | (N = 59) | (N = 46) | (N = 59) | (N = 46) | (95% CI) | P-value¹ |
| MADRS Total | | | | | | |
| at Baseline | | | | | | |
| n | 59 | 46 | | | | |
| Mean (SD) | 32.4 (3.74) | 33.0 (4.39) | | | | |
| Median | 32 | 33 | | | | |
| Min, Max | 27, 42 | 27, 43 | | | | |
| MADRS Total | | | | | | |
| at Week 1 | | | | | | |
| n | 57 | 46 | 57 | 46 | 0.7 (−2.7, 4.1) | 0.702 |
| Mean (SD) | 25.5 (7.31) | 25.5 (8.91) | −6.6 (7.04) | −7.5 (8.85) | | |
| Median | 26 | 26 | −5 | −5 | | |
| Min, Max | 4, 41 | 5, 41 | −30, 3 | −29, 5 | | |
| LSM Estimate | | | −6.7 (1.14) | −7.3 (1.29) | | |
| (SE) | | | | | | |
| MADRS Total | | | | | | |
| at Week 2 | | | | | | |
| n | 52 | 42 | 52 | 42 | −0.7 (−4.1, 2.8) | 0.704 |
| Mean (SD) | 22.6 (9.35) | 23.1 (9.80) | −9.8 (8.94) | −9.8 (9.51) | | |
| Median | 24 | 24 | −9 | −8 | | |
| Min, Max | 1, 40 | 0, 39 | −35, 2 | −33, 3 | | |
| LSM Estimate | | | −10.1 (1.16) | −9.4 (1.31) | | |
| (SE) | | | | | | |
| MADRS Total | | | | | | |
| at Week 3 | | | | | | |
| n | 47 | 40 | 47 | 40 | 2.2 (−1.3, 5.8) | 0.217 |
| Mean (SD) | 21.4 (9.60) | 19.5 (9.25) | −10.6 (9.28) | −13.4 (10.07) | | |
| Median | 22 | 20 | −9 | −12 | | |
| Min, Max | 0, 44 | 0, 38 | −36, 4 | −38, 1 | | |
| LSM Estimate | | | −10.7 (1.20) | −12.9 (1.33) | | |
| (SE) | | | | | | |

Similar results were observed in the other scales, both physician-initiated and patient-initiated, including CGI-S (Tables 6-7), HAM D-17 (Tables 8-9), SDS (Tables 10-11), QIDS-SR (Tables 12-13), and Q-LES-Q-SF (Tables 14-15).

TABLE 6
Female Change from Baseline CGI-S Score Over Time, mITT Population

| | | | Change from Baseline | | | |
|---|---|---|---|---|---|---|
| | SP-624 | Placebo | SP-624 | Placebo | LSM Difference¹ | |
| | (N = 98) | (N = 107) | (N = 98) | (N = 107) | (95% CI) | P-value¹ |
| CGI-S Total | | | | | | |
| at Baseline | | | | | | |
| n | 98 | 107 | | | | |
| Mean (SD) | 4.5 (0.60) | 4.6 (0.57) | | | | |
| Median | 4 | 5 | | | | |
| Min, Max | 4, 6 | 4, 6 | | | | |
| CGI-S Total | | | | | | |
| at Week 1 | | | | | | |
| n | 95 | 105 | 95 | 105 | −0.1 (−0.3, 0.1) | 0.302 |
| Mean (SD) | 4.0 (0.89) | 4.2 (0.90) | −0.5 (0.80) | −0.4 (0.79) | | |
| Median | 4 | 4 | 0 | 0 | | |
| Min, Max | 1, 6 | 1, 6 | −4, 1 | −4, 1 | | |
| LSM Estimate | | | −0.5 (0.08) | −0.4 (0.08) | | |
| (SE) | | | | | | |
| CGI-S Total | | | | | | |
| at Week 2 | | | | | | |
| n | 86 | 92 | 86 | 92 | −0.3 (−0.5, 0.0) | 0.040 |
| Mean (SD) | 3.6 (0.97) | 4.0 (0.95) | −0.8 (0.96) | −0.6 (0.90) | | |
| Median | 4 | 4 | −1 | 0 | | |
| Min, Max | 1, 5 | 2, 6 | −4, 1 | −3, 1 | | |
| LSM Estimate | | | −0.8 (0.10) | −0.6 (0.09) | | |
| (SE) | | | | | | |
| CGI-S Total | | | | | | |
| at Week 3 | | | | | | |
| n | 89 | 90 | 89 | 90 | −0.4 (−0.7, −0.1) | 0.008 |
| Mean (SD) | 3.4 (1.15) | 3.8 (1.03) | −1.1 (1.13) | −0.7 (1.04) | | |
| Median | 4 | 4 | −1 | 0 | | |
| Min, Max | 1, 5 | 1, 5 | −4, 1 | −4, 1 | | |
| LSM Estimate | | | −1.1 (0.11) | −0.7 (0.11) | | |
| (SE) | | | | | | |
| CGI-S Total | | | | | | |
| at Week 4 | | | | | | |
| n | 84 | 86 | 84 | 86 | −0.5 (−0.8, −0.1) | 0.006 |
| Mean (SD) | 3.3 (1.20) | 3.8 (1.10) | −1.2 (1.14) | −0.8 (1.10) | | |
| Median | 3 | 4 | −1 | 0 | | |
| Min, Max | 1, 5 | 1, 5 | −4, 1 | −4, 1 | | |
| LSM Estimate | | | −1.2 (0.12) | −0.8 (0.11) | | |
| (SE) | | | | | | |

TABLE 7
Male Change from Baseline CGI-S Score Over Time, mITT Population

| | | | Change from Baseline | | | |
|---|---|---|---|---|---|---|
| | SP-624 | Placebo | SP-624 | Placebo | LSM Difference¹ | |
| | (N = 59) | (N = 46) | (N = 59) | (N = 46) | (95% CI) | P-value¹ |
| CGI-S Total | | | | | | |
| at Baseline | | | | | | |
| n | 59 | 46 | | | | |
| Mean (SD) | 4.5 (0.60) | 4.6 (0.62) | | | | |
| Median | 4 | 5 | | | | |
| Min, Max | 4, 6 | 4, 6 | | | | |
| CGI-S Total | | | | | | |
| at Week 1 | | | | | | |
| n | 57 | 46 | 57 | 46 | 0.0 (−0.5, 0.4) | 0.827 |
| Mean (SD) | 3.9 (0.84) | 4.0 (0.89) | −0.5 (0.85) | −0.5 (0.89) | | |
| Median | 4 | 4 | 0 | 0 | | |
| Min, Max | 1, 6 | 2, 6 | −4, 1 | −3, 1 | | |
| LSM | | | −0.5 (0.14) | −0.5 (0.16) | | |
| Estimate (SE) | | | | | | |
| CGI-S Total | | | | | | |
| at Week 2 | | | | | | |
| n | 52 | 42 | 52 | 42 | −0.1 (−0.5, 0.3) | 0.566 |
| Mean (SD) | 3.5 (1.18) | 3.7 (1.10) | −0.9 (1.16) | −0.9 (1.15) | | |
| Median | 4 | 4 | −1 | −1 | | |
| Min, Max | 1, 6 | 1, 5 | −4, 0 | −4, 0 | | |
| LSM | | | −1.0 (0.14) | −0.8 (0.16) | | |
| Estimate (SE) | | | | | | |
| CGI-S Total | | | | | | |
| at Week 3 | | | | | | |
| n | 47 | 40 | 47 | 40 | 0.0 (−0.4, 0.4) | 0.996 |
| Mean (SD) | 3.4 (1.15) | 3.4 (1.24) | −1.0 (1.16) | −1.2 (1.33) | | |
| Median | 4 | 4 | −1 | −1 | | |
| Min, Max | 1, 6 | 1, 6 | −4, 1 | −5, 1 | | |
| LSM | | | −1.1 (0.14) | −1.1 (0.16) | | |
| Estimate (SE) | | | | | | |
| CGI-S Total | | | | | | |
| at Week 4 | | | | | | |
| n | 48 | 39 | 48 | 39 | 0.2 (−0.3, 0.6) | 0.437 |
| Mean (SD) | 3.4 (1.27) | 3.3 (1.14) | −1.0 (1.27) | −1.3 (1.26) | | |
| Median | 4 | 3 | −1 | −1 | | |
| Min, Max | 1, 7 | 1, 5 | −4, 2 | −4, 0 | | |
| LSM | | | −1.0 (0.15) | −1.2 (0.16) | | |
| Estimate (SE) | | | | | | |

TABLE 8
Female Change in HAM-D-17 Score Over Time, mITT Population

| | | | Change from Timepoint | | | |
|---|---|---|---|---|---|---|
| | SP-624 | Placebo | SP-624 | Placebo | LSM Difference¹ | |
| | (N = 98) | (N = 107) | (N = 98) | (N = 107) | (95% CI) | P-value¹ |
| HAM-D-17 Total | | | | | | |
| at Baseline | | | | | | |
| n | 98 | 107 | | | | |
| Mean (SD) | 23.2 (3.96) | 23.4 (3.40) | | | | |
| Median | 23 | 24 | | | | |
| Min, Max | 14, 33 | 13, 32 | | | | |
| HAM-D-17 Total | | | | | | |
| at Week 2 | | | | | | |
| n | 86 | 92 | 86 | 92 | −0.9 (−2.6, 0.8) | 0.304 |
| Mean (SD) | 16.6 (6.25) | 17.9 (6.13) | −6.4 (5.99) | −5.2 (6.32) | | |
| Median | 17 | 20 | −5 | −4 | | |
| Min, Max | 1, 30 | 1, 29 | −21, 6 | −28, 4 | | |
| LSM | | | −6.1 (0.63) | −5.2 (0.61) | | |
| Estimate (SE) | | | | | | |
| HAM-D-17 Total | | | | | | |
| at Week 4 | | | | | | |
| n | 84 | 86 | 84 | 86 | −2.3 (−4.2, −0.4) | 0.016 |
| Mean (SD) | 14.6 (7.03) | 16.8 (6.32) | −8.8 (6.82) | −6.3 (6.61) | | |
| Median | 15 | 18 | −8 | −5 | | |
| Min, Max | 0, 27 | 1, 28 | −26, 5 | −28, 4 | | |
| LSM | | | −8.6 (0.68) | −6.3 (0.67) | | |
| Estimate (SE) | | | | | | |

TABLE 9
Male Change in HAM-D-17 Score Over Time, mITT Population

| | | | Change from Timepoint | | | |
|---|---|---|---|---|---|---|
| | SP-624 | Placebo | SP-624 | Placebo | LSM Difference¹ | |
| | (N = 59) | (N = 46) | (N = 59) | (N = 46) | (95% CI) | P-value¹ |
| HAM-D-17 Total | | | | | | |
| at Baseline | | | | | | |
| n | 59 | 46 | | | | |
| Mean (SD) | 21.9 (3.35) | 22.3 (4.19) | | | | |
| Median | 22 | 22 | | | | |
| Min, Max | 15, 29 | 15, 33 | | | | |
| HAM-D-17 Total | | | | | | |
| at Week 2 | | | | | | |
| n | 52 | 42 | 52 | 42 | 0.1 (−2.6, 2.8) | 0.948 |
| Mean (SD) | 16.2 (6.58) | 16.1 (6.76) | −5.7 (5.90) | −6.1 (7.38) | | |
| Median | 17 | 17 | −5 | −4 | | |
| Min, Max | 1, 30 | 0, 28 | −25, 2 | −27, 6 | | |
| LSM | | | −5.9 (0.89) | −6.0 (1.00) | | |
| Estimate (SE) | | | | | | |
| HAM-D-17 Total | | | | | | |
| at Week 4 | | | | | | |
| n | 48 | 39 | 48 | 39 | 1.4 (−1.4, 4.3) | 0.309 |
| Mean (SD) | 14.3 (6.96) | 12.8 (6.54) | −7.8 (6.57) | −9.4 (8.37) | | |
| Median | 15 | 12 | −7 | −8 | | |
| Min, Max | 0, 28 | 1, 27 | −22, 3 | −30, 3 | | |
| LSM | | | −7.7 (0.94) | −9.1 (1.06) | | |
| Estimate (SE) | | | | | | |

TABLE 10
Female Change in SDS Score Over Time, mITT Population

| | | | Change from Baseline | | | |
|---|---|---|---|---|---|---|
| | SP-624 | Placebo | SP-624 | Placebo | LSM Difference¹ | |
| | (N = 98) | (N = 107) | (N = 98) | (N = 107) | (95% CI) | P-value¹ |
| SDS Total | | | | | | |
| at Baseline | | | | | | |
| n | 98 | 106 | | | | |
| Mean (SD) | 16.8 (5.48) | 18.0 (5.64) | | | | |
| Median | 16 | 17 | | | | |
| Min, Max | 3, 30 | 4, 30 | | | | |
| SDS Total at Week 2 | | | | | | |
| n | 86 | 92 | 86 | 92 | −1.7 (−3.3, −0.1) | 0.035 |
| Mean (SD) | 11.8 (6.24) | 14.8 (6.54) | −4.7 (5.74) | −3.2 (5.44) | | |
| Median | 11 | 15 | −4 | −3 | | |
| Min, Max | 0, 29 | 0, 30 | −19, 11 | −18, 10 | | |
| LSM | | | −4.7 (0.58) | −3.0 (0.56) | | |
| Estimate (SE) | | | | | | |
| SDS Total at Week 4 | | | | | | |
| n | 83 | 85 | 83 | 85 | −2.0 (−3.8, −0.2) | 0.029 |
| Mean (SD) | 11.2 (6.98) | 13.6 (7.07) | −5.8 (6.70) | −4.4 (5.91) | | |
| Median | 11 | 14 | −5 | −3 | | |
| Min, Max | 0, 28 | 0, 30 | −22, 14 | −23, 6 | | |
| LSM | | | −5.9 (0.65) | −3.9 (0.64) | | |
| Estimate (SE) | | | | | | |

TABLE 11
Male Change in SDS Score Over Time, mITT Population

| | | | Change from Baseline | | | |
|---|---|---|---|---|---|---|
| | SP-624 | Placebo | SP-624 | Placebo | LSM Difference¹ | |
| | (N = 59) | (N = 46) | (N = 59) | (N = 46) | (95% CI) | P-value¹ |
| SDS Total | | | | | | |
| at Baseline | | | | | | |
| n | 59 | 46 | | | | |
| Mean (SD) | 15.3 (5.01) | 16.0 (5.85) | | | | |
| Median | 15 | 16 | | | | |
| Min, Max | 3, 30 | 8, 30 | | | | |
| SDS Total at Week 2 | | | | | | |
| n | 52 | 42 | 52 | 42 | 2.6 (0.3, 4.8) | 0.025 |
| Mean (SD) | 12.8 (5.99) | 10.7 (6.58) | −2.4 (5.27) | −5.3 (6.37) | | |
| Median | 13 | 10 | −2 | −4 | | |
| Min, Max | 1, 30 | 0, 30 | −14, 10 | −20, 9 | | |
| LSM | | | −2.6 (0.75) | −5.1 (0.84) | | |
| Estimate (SE) | | | | | | |
| SDS Total at Week 4 | | | | | | |
| n | 48 | 39 | 48 | 39 | 1.3 (−1.3, 3.8) | 0.320 |
| Mean (SD) | 10.5 (6.39) | 9.7 (7.38) | −5.0 (6.14) | −6.5 (7.11) | | |
| Median | 10 | 10 | −4 | −7 | | |
| Min, Max | 0, 30 | 0, 30 | −18, 7 | −20, 8 | | |
| LSM | | | −4.9 (0.85) | −6.2 (0.96) | | |
| Estimate (SE) | | | | | | |

TABLE 12
Female Change in QIDS Score Over Time, mITT Population

| | | | Change from Baseline | | | |
|---|---|---|---|---|---|---|
| | SP-624 | Placebo | SP-624 | Placebo | LSM Difference¹ | |
| | (N = 98) | (N = 107) | (N = 98) | (N = 107) | (95% CI) | P-value¹ |
| QIDS Total | | | | | | |
| at Baseline | | | | | | |
| n | 98 | 107 | | | | |
| Mean (SD) | 15.8 (3.67) | 16.5 (3.76) | | | | |
| Median | 16 | 17 | | | | |
| Min, Max | 6, 25 | 8, 25 | | | | |
| QIDS Total | | | | | | |
| at Week 2 | | | | | | |
| n | 86 | 92 | 86 | 92 | −1.1 (−2.4, 0.3) | 0.118 |
| Mean (SD) | 11.7 (4.87) | 13.5 (5.24) | −3.9 (4.37) | −3.0 (5.10) | | |
| Median | 12 | 14 | −3 | −2 | | |
| Min, Max | 1, 21 | 2, 24 | −16, 5 | −21, 6 | | |
| LSM | | | −3.9 (0.49) | −2.9 (0.47) | | |
| Estimate (SE) | | | | | | |
| QIDS Total | | | | | | |
| at Week 4 | | | | | | |
| n | 83 | 85 | 83 | 85 | −2.0 (−3.3, −0.6) | 0.004 |
| Mean (SD) | 10.2 (4.69) | 12.4 (5.05) | −5.7 (4.76) | −4.2 (5.07) | | |
| Median | 10 | 13 | −5 | −3 | | |
| Min, Max | 0, 19 | 1, 22 | −20, 4 | −22, 4 | | |
| LSM | | | −5.9 (0.48) | −3.9 (0.47) | | |
| Estimate (SE) | | | | | | |

TABLE 13
Male Change in QIDS Score Over Time, mITT Population

| | | | Change from Baseline | | | |
|---|---|---|---|---|---|---|
| | SP-624 | Placebo | SP-624 | Placebo | LSM Difference¹ | |
| | (N = 59) | (N = 46) | (N = 59) | (N = 46) | (95% CI) | P-value¹ |
| QIDS Total | | | | | | |
| at Baseline | | | | | | |
| n | 59 | 46 | | | | |
| Mean (SD) | 13.8 (4.08) | 14.8 (3.67) | | | | |
| Median | 14 | 15 | | | | |
| Min, Max | 6, 23 | 7, 24 | | | | |
| QIDS Total | | | | | | |
| at Week 2 | | | | | | |
| n | 52 | 42 | 52 | 42 | 1.2 (−0.9, 3.2) | 0.258 |
| Mean (SD) | 11.7 (5.27) | 11.1 (5.38) | −2.1 (5.50) | −3.8 (4.98) | | |
| Median | 12 | 12 | −2 | −2 | | |
| Min, Max | 1, 24 | 0, 24 | −17, 11 | −17, 4 | | |
| LSM | | | −2.3 (0.68) | −3.5 (0.76) | | |
| Estimate (SE) | | | | | | |
| QIDS Total | | | | | | |
| at Week 4 | | | | | | |
| n | 48 | 39 | 48 | 39 | 1.1 (−0.8, 3.0) | 0.247 |
| Mean (SD) | 10.4 (4.55) | 10.1 (5.42) | −3.3 (4.46) | −5.1 (5.06) | | |
| Median | 11 | 10 | −4 | −4 | | |
| Min, Max | 0, 19 | 2, 23 | −15, 6 | −17, 4 | | |
| LSM | | | −3.5 (0.63) | −4.6 (0.71) | | |
| Estimate (SE) | | | | | | |

TABLE 14
Female Change in Q-LES-Q-SF Score Over Time, mITT Population

| | | | Change from Baseline | | | |
|---|---|---|---|---|---|---|
| | SP-624 | Placebo | SP-624 | Placebo | LSM Difference¹ | |
| | (N = 98) | (N = 107) | (N = 98) | (N = 107) | (95% CI) | P-value¹ |
| Q-LES-Q-SF Total | | | | | | |
| at Baseline | | | | | | |
| n | 98 | 107 | | | | |
| Mean (SD) | 50.9 (9.91) | 51.1 (10.37) | | | | |
| Median | 51 | 51 | | | | |
| Min, Max | 24, 73 | 24, 77 | | | | |
| Q-LES-Q-SF Total | | | | | | |
| at Week 2 | | | | | | |
| n | 86 | 92 | 86 | 92 | 1.1 (−1.8, 3.9) | 0.459 |
| Mean (SD) | 59.2 (11.32) | 57.2 (12.43) | 7.8 (10.32) | 6.3 (9.72) | | |
| Median | 59 | 56 | 6 | 6 | | |
| Min, Max | 36, 84 | 27, 83 | −11, 34 | −14, 42 | | |
| LSM | | | 7.4 (1.03) | 6.4 (1.00) | | |
| Estimate (SE) | | | | | | |
| Q-LES-Q-SF Total | | | | | | |
| at Week 4 | | | | | | |
| n | 83 | 85 | 83 | 85 | 3.3 (−0.3, 6.8) | 0.074 |
| Mean (SD) | 62.1 (13.87) | 58.8 (14.01) | 11.4 (14.11) | 7.6 (10.67) | | |
| Median | 59 | 59 | 10 | 6 | | |
| Min, Max | 33, 99 | 23, 100 | −11, 53 | −16, 50 | | |
| LSM | | | 11.0 (1.29) | 7.7 (1.27) | | |
| Estimate (SE) | | | | | | |

TABLE 15
Male Change in Q-LES-Q-SF Score Over Time, mITT Population

| | | | Change from Baseline | | | |
|---|---|---|---|---|---|---|
| | SP-624 | Placebo | SP-624 | Placebo | LSM Difference¹ | |
| | (N = 59) | (N = 46) | (N = 59) | (N = 46) | (95% CI) | P-value¹ |
| Q-LES-Q-SF Total | | | | | | |
| at Baseline | | | | | | |
| n | 59 | 46 | | | | |
| Mean (SD) | 54.4 (9.39) | 52.3 (11.24) | | | | |
| Median | 54 | 54 | | | | |
| Min, Max | 27, 79 | 31, 73 | | | | |
| Q-LES-Q-SF Total | | | | | | |
| at Week 2 | | | | | | |
| n | 52 | 42 | 52 | 42 | −2.2 (−6.7, 2.3) | 0.331 |
| Mean (SD) | 59.5 (12.80) | 61.2 (13.68) | 5.5 (10.39) | 7.9 (12.12) | | |
| Median | 59 | 61 | 4 | 6 | | |
| Min, Max | 20, 94 | 33, 91 | −17, 37 | −13, 37 | | |
| LSM | | | 5.6 (1.50) | 7.8 (1.68) | | |
| Estimate (SE) | | | | | | |
| Q-LES-Q-SF Total | | | | | | |
| at Week 4 | | | | | | |
| n | 48 | 39 | 48 | 39 | −1.3 (−6.4, 3.7) | 0.603 |
| Mean (SD) | 61.5 (12.26) | 61.2 (15.51) | 7.1 (11.54) | 8.6 (14.17) | | |
| Median | 61 | 63 | 6 | 6 | | |
| Min, Max | 36, 96 | 26, 91 | −11, 43 | −16, 42 | | |
| LSM | | | 6.9 (1.70) | 8.2 (1.91) | | |
| Estimate (SE) | | | | | | |

The foregoing examples are illustrative of the present invention and are not to be construed as limiting thereof. Although the invention has been described in detail with reference to preferred embodiments, variations and modifications exist within the scope and spirit of the invention as described and defined in the following claims.

1. (canceled)

2. A method of treating depression in a human female subject in need thereof, comprising identifying the subject as female and administering to the identified female subject a therapeutically effective amount of a SIRT6 activator, thereby treating the depression.

3. A method of treating depression in a human subject in need thereof, comprising:
a) determining the gender of the subject; and
b) administering to the subject a therapeutically effective amount of a SIRT6 activator if the subject is determined to be female, thereby treating the depression, or not administering to the subject a therapeutically effective amount of a SIRT6 activator if the subject is determined to be male.

4. The method of claim 2, wherein identifying the subject as female or determining the gender of the subject comprises determining whether the subject has two X chromosomes.

5. The method of claim 2, wherein identifying the subject as female or determining the gender of the subject comprises determining the level of circulating female hormones in the subject.

6. The method of claim 2, wherein the depression is major depressive disorder, persistent depressive disorder, minor depression, treatment-resistant depression, substance/medication-induced depression, depressive disorder secondary to medical illness, perinatal and postpartum depression, postmenopausal depression, premenstrual dysphoric disorder, seasonal affective disorder, psychotic depression, or bipolar disorder.

7. The method of claim 2, wherein the SIRT6 activator is quercetin, isoquercetin, kaempferol, luteolin, cyanidin, fisetin, delphinidin, icariin, N-acetylethanolamines, oleic acid, linoleic acid, fucoidan, MDL-800, MDL-811, UBCS038, UBCS039, UBCS040, UBCS058, UBCS060, UBCS068, myristic acid, OEA, CL5D, or 10b.

8. The method of claim 2, wherein the SIRT6 activator is a compound of Formula 1 or a pharmaceutically acceptable salt thereof: 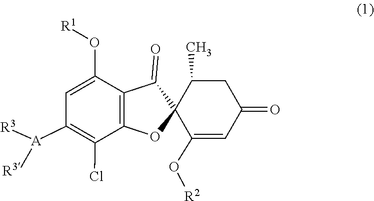
wherein:
R¹ is a C1-C6 alkyl group optionally substituted with the same or different one to two substituents selected from a substituent group X,
a C3-C6 cycloalkyl group optionally substituted with the same or different one to two substituents selected from the substituent group X, or
a 4-7 membered saturated heterocyclic group optionally substituted with the same or different one to two substituents selected from the substituent group X,
R² is a C1-C6 alkyl group optionally substituted with the same or different one to two substituents selected from the substituent group X,
a C3-C6 cycloalkyl group optionally substituted with the same or different one to two substituents selected from the substituent group X, or
a 4-7 membered saturated heterocyclic group optionally substituted with the same or different one to two substituents selected from the substituent group X,
A is a 5-membered aromatic heterocyclic ring,
a 6-membered aromatic heterocyclic ring,
an 8-10 membered condensed aromatic heterocyclic ring,
a 5-7 membered unsaturated heterocyclic ring,
a 4-7 membered saturated heterocyclic ring,
a benzene ring, —CH—, or a cyano group, wherein when A is a cyano group, R³ and R^{3′} do not exist, R³ and R³′ are each independently a hydrogen atom, a halogen atom, a cyano group, a hydroxy group, an oxo group,
a C1-C6 alkyl group optionally substituted with the same or different one to two substituents selected from the substituent group X,
a C1-C6 alkoxy group optionally substituted with the same or different one to two substituents selected from the substituent group X,
a C2-C6 alkenyl group optionally substituted with the same or different one to two substituents selected from the substituent group X,
a C2-C6 alkynyl group optionally substituted with the same or different one to two substituents selected from the substituent group X,
a C3-C6 cycloalkyl group optionally substituted with the same or different one to two substituents selected from the substituent group X,
an amino group optionally substituted with the same or different one to two substituents selected from the substituent group X,
a C1-C6 alkoxycarbonyl group optionally substituted with the same or different one to two substituents selected from the substituent group X,
a carbamoyl group optionally substituted with the same or different one to two substituents selected from the substituent group X,
a phenyl group optionally substituted with the same or different one to two substituents selected from the substituent group X,
a 5-membered aromatic heterocyclic group optionally substituted with the same or different one to two substituents selected from the substituent group X,
a 6-membered aromatic heterocyclic group optionally substituted with the same or different one to two substituents selected from the substituent group X,
a 5-7 membered unsaturated heterocyclic group optionally substituted with the same or different one to two substituents selected from the substituent group X,
a 4-7 membered saturated heterocyclic group optionally substituted with the same or different one to two substituents selected from the substituent group X,
an 8-10 membered condensed aromatic heterocyclic group optionally substituted with the same or different one to two substituents selected from the substituent group X, or
R³ and R^{3′} may form a 5-7 membered unsaturated heterocyclic ring, a 4-7 membered saturated heterocyclic ring, or a C3-C6 cycloalkyl ring as a ring that binds to each other and condenses with A, and the ring is optionally substituted with the same or different one to two substituents selected from the substituent group X,
substituent group X is a halogen atom, a cyano group, a hydroxy group, an oxo group, a C1-C6 alkyl group, a hydroxy C1-C6 alkyl group, a C1-C6 alkoxy C1-C6 alkyl group, a C1-C6 haloalkyl group, a C3-C6 cycloalkyl group, a C3-C6 halocycloalkyl group,
a phenyl group optionally substituted with the same or different one to two substituents selected from a substituent group Y,
a 5-membered aromatic heterocyclic group optionally substituted with the same or different one to two substituents selected from the substituent group Y,
a 6-membered aromatic heterocyclic group optionally substituted with the same or different one to two substituents selected from the substituent group Y,
a 4-7 membered saturated heterocyclic group optionally substituted with the same or different one to two substituents selected from the substituent group Y,
a C1-C6 alkoxy group, a C1-C6 haloalkoxy group, a C3-C6 cycloalkoxy group, a C3-C6 halocycloalkoxy group,
a phenoxy group optionally substituted with the same or different one to two substituents selected from the substituent group Y,
a 5-membered aromatic heterocyclic oxy group optionally substituted with the same or different one to two substituents selected from the substituent group Y,
a 6-membered aromatic heterocyclic oxy group optionally substituted with the same or different one to two substituents selected from the substituent group Y,
a 4-7 membered saturated heterocyclic oxy group optionally substituted with the same or different one to two substituents selected from the substituent group Y,
a C1-C6 alkoxycarbonyl group, a C3-C6 cycloalkoxycarbonyl group, a carboxy group, a C1-C6 alkylcarbonyl group, a C3-C6 cycloalkylcarbonyl group,
a phenylcarbonyl group optionally substituted with the same or different one to two substituents selected from the substituent group Y,
a carbamoyl group, a mono (C1-C6 alkyl)aminocarbonyl group, a di (C1-C6 alkyl)aminocarbonyl group, a mono (C1-C6 alkyl)aminosulfonyl group, a di (C1-C6 alkyl)aminosulfonyl group, an amino group, a mono (C1-C6 alkyl) amino group, a di (C1-C6 alkyl) amino group, a C1-C6 alkoxycarbonylamino group, a mono (C1-C6 alkyl)aminocarbonylamino group, a di (C1-C6 alkyl)aminocarbonylamino group, a C1-C6 alkylcarbonylamino group,
a phenylcarbonylamino group optionally substituted with the same or different one to two substituents selected from the substituent group Y,
a 5-membered aromatic heterocyclic carbonylamino group optionally substituted with the same or different one to two substituents selected from the substituent group Y,
a 6-membered aromatic heterocyclic carbonylamino group optionally substituted with the same or different one to two substituents selected from the substituent group Y, or a C1-C6 alkylsulfonylamino group,
substituent group Y is a C1-C6 alkyl group, a C1-C6 alkoxy group, a halogen atom, or a hydroxy group,
thereby improving memory function, cognition function, and/or learning function in the subject.

9. The method of claim 8, wherein the 5-membered aromatic heterocyclic ring or the 5-membered aromatic heterocyclic group in A, R³, or R³′ is any one selected from the group:

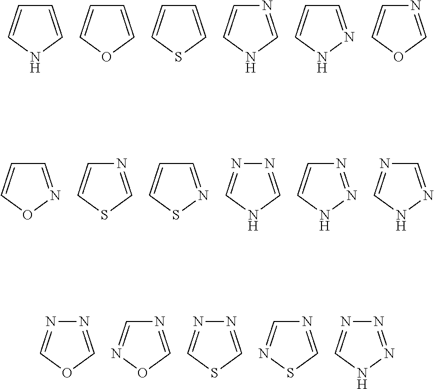

10. The method of claim 8, wherein the 6-membered aromatic heterocyclic ring or the 6-membered aromatic heterocyclic group in A, R³, or R³′ is any one selected from the group:

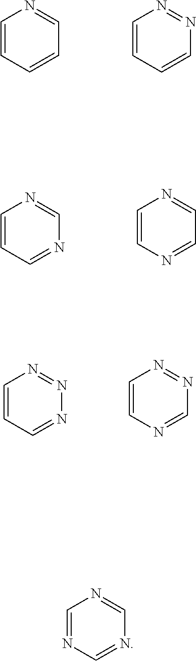

11. The method of claim 8, wherein the 8-10 membered condensed aromatic heterocyclic ring or the 8-10 membered condensed aromatic heterocyclic group in A, R³, or R³′ is any one selected from the group:

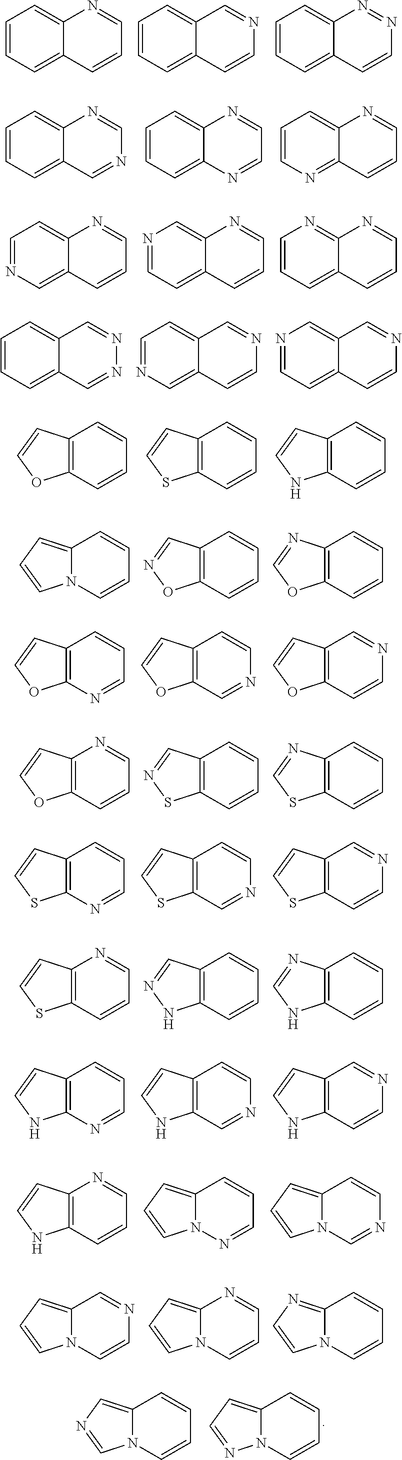

12. The method of claim 8, wherein the 5-7 membered unsaturated heterocyclic ring or 5-7 membered unsaturated heterocyclic group in A, R³, or R³′ is any one selected from the group:

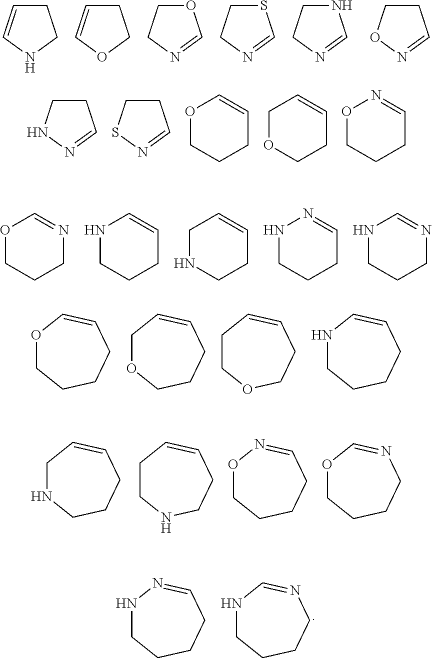

13. The method of claim 8, wherein the 4-7 membered saturated heterocyclic ring or the 4-7 membered saturated heterocyclic group in A, R¹, R², or R³ is any one selected from the group:

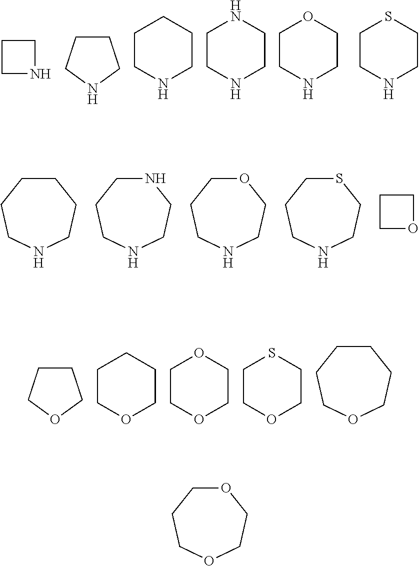

14. The method of claim 8, wherein the compound of Formula 1 is any compound selected from the following group:
(2S,5′R)-7-chloro-6-(5-ethyl-1,3,4-oxadiazol-2-yl)-3′,4-dimethoxy-5′-methyl-spiro[benzofuran-2,4′-cyclohex-2-ene]-1′,3-dione;
(2S,5′R)-7-chloro-3′,4-dimethoxy-5′-methyl-6-(5-tetrahydropyran-4-yl-1,3,4-oxadiazol-2-yl) spiro[benzofuran-2,4′-cyclohex-2-ene]-1′,3-dione;
(2S,5′R)-7-chloro-3′,4-dimethoxy-5′-methyl-6-[5-(1-methyl-4-piperidyl)-1,3,4-oxadiazol-2-yl] spiro[benzofuran-2,4′-cyclohex-2-ene]-1′,3-dione;
(2S,5′R)-7-chloro-6-[5-(4-fluoro-1-methyl-4-piperidyl)-1,3,4-oxadiazol-2-yl]-3′,4-dimethoxy-5′-methyl-spiro[benzofuran-2,4′-cyclohex-2-ene]-1′,3-dione;
(2S,5′R)-7-chloro-3′,4-dimethoxy-6-[5-[(1S)-1-methoxyethyl]-1,3,4-oxadiazol-2-yl]-5′-methyl-spiro[benzofuran-2,4′-cyclohex-2-ene]-1′,3-dione;
(2S,5′R)-7-chloro-4-ethoxy-3′-methoxy-5′-methyl-6-(5-methyl-1,3,4-oxadiazol-2-yl) spiro[benzofuran-2,4′-cyclohex-2-ene]-1′,3-dione;
(2S,5′R)-7-chloro-4-(difluoromethoxy)-3′-methoxy-5′-methyl-6-(5-methyl-1,3,4-oxadiazol-2-yl) spiro[benzofuran-2,4′-cyclohex-2-ene]-1′,3-dione;
(2S,5′R)-7-chloro-3′,4-dimethoxy-6-[3-(1-methoxyethyl)-1,2,4-oxadiazol-5-yl]-5′-methyl-spiro[benzofuran-2,4′-cyclohex-2-ene]-1′,3-dione;
(2S,5′R)-7-chloro-6-[3-(1-hydroxy-1-methyl-ethyl)-1,2,4-oxadiazol-5-yl]-3′,4-dimethoxy-5′-methyl-spiro[benzofuran-2,4′-cyclohex-2-ene]-1′,3-dione;
(2S,5′R)-7-chloro-3′,4-dimethoxy-5′-methyl-6-(1H-pyrazol-5-yl) spiro[benzofuran-2,4′-cyclohex-2-ene]-1′,3-dione;
(2S,5′R)-7-chloro-3′,4-dimethoxy-6-[1-(2-methoxyethyl) pyrazol-3-yl]-5′-methyl-spiro[benzofuran-2,4′-cyclohex-2-ene]-1′,3-dione;
(2S,5′R)-7-chloro-6-(1,8-dioxa-2-azaspiro[4.5]dec-2-en-3-yl)-3′,4-dimethoxy-5′-methyl-spiro[benzofuran-2,4′-cyclohex-2-ene]-1′,3-dione;
(2S,5′R)-7-chloro-3′,4-dimethoxy-5′-methyl-6-(8-methyl-1-oxa-2,8-diazaspiro[4.5] dec-2-en-3-yl) spiro[benzofuran-2,4′-cyclohex-2-ene]-1′,3-dione;
(2S,5′R)-7-chloro-3′,4-dimethoxy-6-(2-methoxypyrimidin-5-yl)-5′-methyl-spiro[benzofuran-2,4′-cyclohex-2-ene]-1′,3-dione;
(2S,5′R)-7-chloro-3′,4-dimethoxy-6-(6-methoxy-3-pyridyl)-5′-methyl-spiro[benzofuran-2,4′-cyclohex-2-ene]-1′,3-dione; or
(2S,5′R)-7-chloro-3′,4-dimethoxy-5′-methyl-6-(3-pyridyl) spiro[benzofuran-2,4′-cyclohex-2-ene]-1′,3-dione.

15. The method of claim 8, wherein the compound of Formula 1 is a compound of Formula 1′ or a pharmacologically acceptable salt thereof: 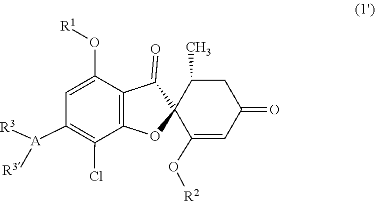
wherein:
R¹ is a C1-C6 alkyl group optionally substituted with the same or different one to two substituents selected from the substituent group X,
a C3-C6 cycloalkyl group optionally substituted with the same or different one to two substituents selected from the substituent group X, or
a 4-7 membered saturated heterocyclic group optionally substituted with the same or different one to two substituents selected from the substituent group X
R² is a C1-C6 alkyl group optionally substituted with the same or different one to two substituents selected from the substituent group X,
a C3-C6 cycloalkyl group optionally substituted with the same or different one to two substituents selected from the substituent group X, or
a 4-7 membered saturated heterocyclic group optionally substituted with the same or different one to two substituents selected from the substituent group X.
A is a 5-membered aromatic heterocyclic ring,
a 6-membered aromatic heterocyclic ring,
an 8-10 membered condensed aromatic heterocyclic ring,
a 5-7 membered unsaturated heterocyclic ring,
a 4-7 membered saturated heterocyclic ring,
a benzene ring, or a single bond, wherein when it is a single bond, one or the other of R³ and R³′ is not present,
R³ and R³′ are each independently a hydrogen atom, a halogen atom, a cyano group, a hydroxy group,
a C1-C6 alkyl group optionally substituted with the same or different one to two substituents selected from the substituent group X,
a C1-C6 alkoxy group optionally substituted with the same or different one to two substituents selected from the substituent group X,
a C2-C6 alkenyl group optionally substituted with the same or different one to two substituents selected from the substituent group X,
a C2-C6 alkynyl group optionally substituted with the same or different one to two substituents selected from the substituent group X,
a C3-C6 cycloalkyl group optionally substituted with the same or different one to two substituents selected from the substituent group X,
an amino group optionally substituted with the same or different one to two substituents selected from the substituent group X,
a C1-C6 alkoxycarbonyl group optionally substituted with the same or different one to two substituents selected from the substituent group X,
a carbamoyl group optionally substituted with the same or different one to two substituents selected from the substituent group X,
a phenyl group optionally substituted with the same or different one to two substituents selected from the substituent group X,
a 5-membered aromatic heterocyclic group optionally substituted with the same or different one to two substituents selected from the substituent group X,
a 6-membered aromatic heterocyclic group optionally substituted with the same or different one to two substituents selected from the substituent group X,
a 5-7 membered unsaturated heterocyclic group optionally substituted with the same or different one to two substituents selected from the substituent group X,
a 4-7 membered saturated heterocyclic group optionally substituted with the same or different one to two substituents selected from the substituent group X,
an 8-10 membered condensed aromatic heterocyclic group optionally substituted with the same or different one to two substituents selected from the substituent group X,
or
R³ and R³′ may form a 5-7 membered unsaturated heterocyclic ring, a 4-7 membered saturated heterocyclic ring, or a C3-C6 cycloalkyl ring as a ring that binds to each other and condenses with A, and the ring is optionally substituted with the same or different one to two substituents selected from the substituent group X,
Substituent group X is a halogen atom, a cyano group, a hydroxy group, an oxo group, a C1-C6 alkyl group, a hydroxy C1-C6 alkyl group, a C1-C6 alkoxy C1-C6 alkyl group, a C1-C6 haloalkyl group, a C3-C6 cycloalkyl group, a C3-C6 halocycloalkyl group,
a phenyl group optionally substituted with the same or different one to two substituents selected from the substituent group Y,
a 5-membered aromatic heterocyclic group optionally substituted with the same or different one to two substituents selected from the substituent group Y,
a 6-membered aromatic heterocyclic group optionally substituted with the same or different one to two substituents selected from the substituent group Y,
a 4-7 membered saturated heterocyclic group optionally substituted with the same or different one to two substituents selected from the substituent group Y,
a C1-C6 alkoxy group, a C1-C6 haloalkoxy group, a C3-C6 cycloalkoxy group, a C3-C6 halocycloalkoxy group,
a phenoxy group optionally substituted with the same or different one to two substituents selected from the substituent group Y,
a 5-membered aromatic heterocyclic oxy group optionally substituted with the same or different one to two substituents selected from the substituent group Y,
a 6-membered aromatic heterocyclic oxy group optionally substituted with the same or different one to two substituents selected from the substituent group Y,
a 4-7 membered saturated heterocyclic oxy group optionally substituted with the same or different one to two substituents selected from the substituent group Y,
a C1-C6 alkoxycarbonyl group, a C3-C6 cycloalkoxycarbonyl group, a carboxy group, a C1-C6 alkylcarbonyl group, a C3-C6 cycloalkylcarbonyl group,
a phenylcarbonyl group optionally substituted with the same or different one to two substituents selected from the substituent group Y,
a carbamoyl group, a mono (C1-C6 alkyl)aminocarbonyl group, a di (C1-C6 alkyl)aminocarbonyl group, a mono (C1-C6 alkyl)aminosulfonyl group, a di (C1-C6 alkyl)aminosulfonyl group, an amino group, a mono (C1-C6 alkyl) amino group, a di (C1-C6 alkyl) amino group, a C1-C6 alkoxycarbonylamino group, a mono (C1-C6 alkyl)aminocarbonylamino group, a di (C1-C6 alkyl)aminocarbonylamino group, a C1-C6 alkylcarbonylamino group,
a phenylcarbonylamino group optionally substituted with the same or different one to two substituents selected from the substituent group Y,
a 5-membered aromatic heterocyclic carbonylamino group optionally substituted with the same or different one to two substituents selected from the substituent group Y,
a 6-membered aromatic heterocyclic carbonylamino group optionally substituted with the same or different one to two substituents selected from the substituent group Y, or
a C1-C6 alkylsulfonylamino group, and
Substituent group Y is a C1-C6 alkyl group, a C1-C6 alkoxy group, a halogen atom, or a hydroxy group.

16. The method of claim 15, wherein R¹ is a methyl group, an ethyl group, or a hydroxyethyl group.

17. The method of claim 15, wherein R² is a methyl group.

18. The method of claim 15, wherein the 5-membered aromatic heterocyclic ring or the 5-membered aromatic heterocyclic group in A, R³, or R³′ is any one selected from the group:

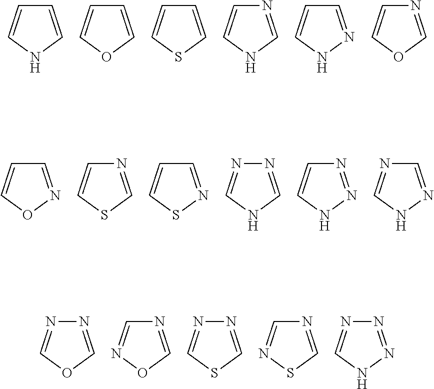

19. The method of claim 15, wherein the 6-membered aromatic heterocyclic ring or the 6-membered aromatic heterocyclic group in A, R³, or R³′ is any one selected from the group:

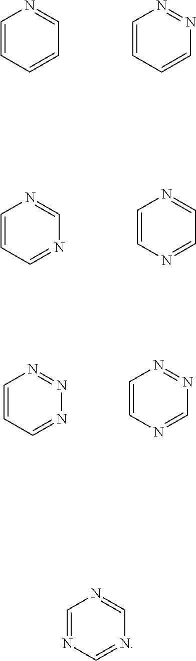

20. The method of claim 15, wherein the 5-7 membered unsaturated heterocyclic ring or the 5-7 membered unsaturated heterocyclic group in A, R³, or R³′ is any one selected from the group:

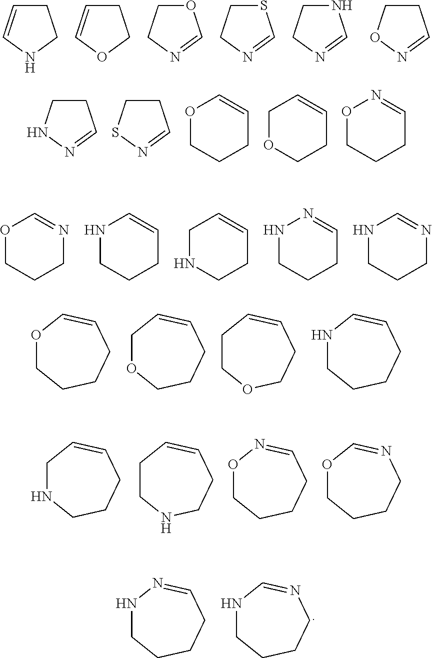

21. The method of claim 15, wherein the 4-7 membered saturated heterocyclic ring or the 4-7 membered saturated heterocyclic group in A, R¹, R², or R³ is any one selected from the group:

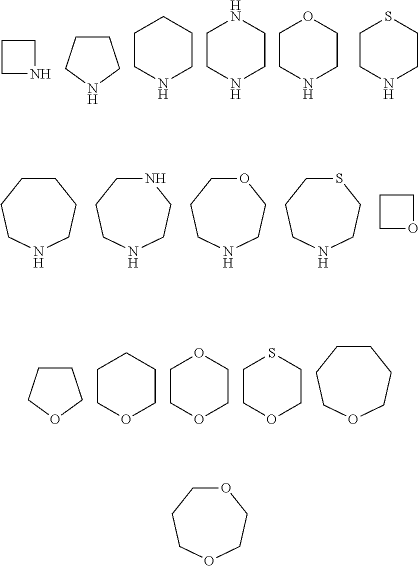

22. The method of claim 15, wherein A is a 5-membered aromatic heterocyclic ring, R³ is a methyl group, an ethyl group, a hydroxy C1-C3 alkyl group, or a methoxy C1-C3 alkyl group, and R³′ is a hydrogen atom.

23. The method of claim 15, wherein A is any ring selected from the following group, and in the case of two binding groups, R³′ is not present: 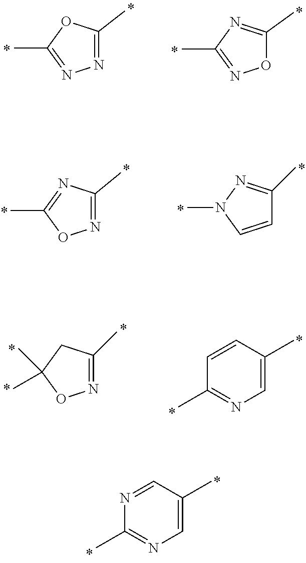
wherein * indicates a binding group.

24. The method of claim 15, wherein the compound of Formula 1 is a compound of a Formula 1″ or a pharmacologically acceptable salt thereof: 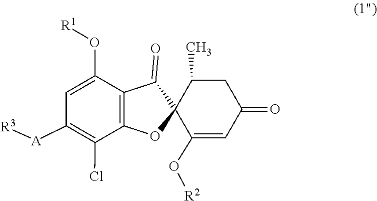
wherein R¹ is a methyl group or an ethyl group;
R² is a methyl group;
A is any ring selected from the following group: 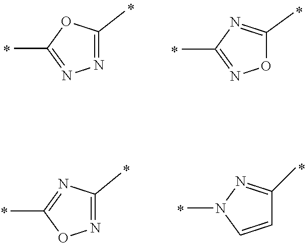
* indicates a binding group; and
R³ is a methyl group or an ethyl group.

25. The method of claim 15, wherein the compound of Formula 1′ is any compound selected from the following group:
(2S,5′R)-7-chloro-6-(2-hydroxyethoxy)-3′,4-dimethoxy-5′-methyl-spiro[benzofuran-2,4′-cyclohex-2-ene]-1′,3-dione;
(2S,5′R)-7-chloro-3′,4-dimethoxy-6-(2-methoxyethoxy)-5′-methyl-spiro[benzofuran-2,4′-cyclohex-2-ene]-1′,3-dione;
(2S,5′R)-7-chloro-3′,4-dimethoxy-5′-methyl-6-(1-methylpyrazol-3-yl) spiro[benzofuran-2,4′-cyclohex-2-ene]-1′,3-dione;
(2S,5′R)-7-chloro-6-(1-ethylpyrazol-3-yl)-3′,4-dimethoxy-5′-methyl-spiro[benzofuran-2,4′-cyclohex-2-ene]-1′,3-dione;
(2S,5′R)-7-chloro-3′,4-dimethoxy-5′-methyl-6-(5-methyl-1,3,4-oxadiazol-2-yl) spiro[benzofuran-2,4′-cyclohex-2-ene]-1′,3-dione;
(2S,5′R)-7-chloro-3′,4-dimethoxy-5′-methyl-6-(3-methyl-1,2,4-oxadiazol-5-yl) spiro[benzofuran-2,4′-cyclohex-2-ene]-1′,3-dione;
(2S,5′R)-7-chloro-3′,4-dimethoxy-5′-methyl-6-(5-methyl)-1,2,4-oxadiazol-3-yl) spiro [benzofuran-2,4′-cyclohex-2-ene]-1′,3-dione;
(2S,5′R)-7-chloro-6-[5-(1-hydroxy-1-methyl-ethyl)-1,3,4-oxadiazol-2-yl]-3′,4-dimethoxy-5′-methyl-spiro[benzofuran-2,4′-cyclohex-2-ene]-1′,3-dione;
(2S,5′R)-7-chloro-6-[5-[(1S)-1-hydroxyethyl]-1,3,4-oxadiazol-2-yl]-3′,4-dimethoxy-5′-methyl-spiro[benzofuran-2,4′-cyclohex-2-ene]-1′,3-dione;
(2S,5′R)-7-chloro-6-[5-[(1R)-1-hydroxyethyl]-1,3,4-oxadiazol-2-yl]-3′,4-dimethoxy-5′-methyl-spiro[benzofuran-2,4′-cyclohex-2-ene]-1′,3-dione;
(2S,5′R)-7-chloro-4-ethoxy-6-[5-(1-hydroxy-1-methyl-ethyl)-1,3,4-oxadiazol-2-yl]-3′-methoxy-5′-methyl-spiro[benzofuran-2,4′-cyclohex-2-ene]-1′,3-dione;
(2S,5′R)-7-chloro-4-ethoxy-6-[5-[(1S)-1-hydroxyethyl]-1,3,4-oxadiazol-2-yl]-3′-methoxy-5′-methyl-spiro[benzofuran-2,4′-cyclohex-2-ene]-1′,3-dione;
(2S,5′R)-7-chloro-6-[3-(1-hydroxyethyl)-1,2,4-oxadiazol-5-yl]-3′,4-dimethoxy-5′-methyl-spiro[benzofuran-2,4′-cyclohex-2-ene]-1′,3-dione;
(2S,5′R)-7-chloro-4-(2-hydroxyethoxy)-3′-methoxy-5′-methyl-6-(5-methyl-1,3,4-oxadiazol-2-yl) spiro[benzofuran-2,4′-cyclohex-2-ene]-1′,3-dione;
(2S,5′R)-7-chloro-4-(2-hydroxyethoxy)-3′-methoxy-5′-methyl-6-(3-methyl-1,2,4-oxadiazol-5-yl) spiro[benzofuran-2,4′-cyclohex-2-ene]-1′,3-dione;
(2S,5′R)-7-chloro-4-(2-hydroxyethoxy)-3′-methoxy-5′-methyl-6-(5-methyl-1,2,4-oxadiazol-3-yl) spiro[benzofuran-2,4′-cyclohex-2-ene]-1′,3-dione;
(2S,5′R)-7-chloro-6-(5-ethyl-1,3,4-oxadiazol-2-yl)-3′,4-dimethoxy-5′-methyl-spiro[benzofuran-2,4′-cyclohex-2-ene]-1′,3-dione;
(2S,5′R)-7-chloro-3′,4-dimethoxy-5′-methyl-6-(5-tetrahydropyran-4-yl-1,3,4-oxadiazol-2-yl) spiro[benzofuran-2,4′-cyclohex-2-ene]-1′,3-dione;
(2S,5′R)-7-chloro-3′,4-dimethoxy-5′-methyl-6-[5-(1-methyl-4-piperidyl)-1,3,4-oxadiazol-2-yl] spiro[benzofuran-2,4′-cyclohex-2-ene]-1′,3-dione;
(2S,5′R)-7-chloro-6-[5-(4-fluoro-1-methyl-4-piperidyl)-1,3,4-oxadiazol-2-yl]-3′,4-dimethoxy-5′-methyl-spiro[benzofuran-2,4′-cyclohex-2-ene]-1′,3-dione;
(2S,5′R)-7-chloro-3′,4-dimethoxy-6-[5-[(1S)-1-methoxyethyl]-1,3,4-oxadiazol-2-yl]-5′-methyl-spiro[benzofuran-2,4′-cyclohex-2-ene]-1′,3-dione;
(2S,5′R)-7-chloro-4-ethoxy-3′-methoxy-5′-methyl-6-(5-methyl-1,3,4-oxadiazol-2-yl) spiro[benzofuran-2,4′-cyclohex-2-ene]-1′,3-dione;
(2S,5′R)-7-chloro-4-(difluoromethoxy)-3′-methoxy-5′-methyl-6-(5-methyl-1,3,4-oxadiazol-2-yl) spiro[benzofuran-2,4′-cyclohex-2-ene]-1′,3-dione;
(2S,5′R)-7-chloro-3′,4-dimethoxy-6-[3-(1-methoxyethyl)-1,2,4-oxadiazol-5-yl]-5′-methyl-spiro[benzofuran-2,4′-cyclohex-2-ene]-1′,3-dione;
(2S,5′R)-7-chloro-6-[3-(1-hydroxy-1-methyl-ethyl)-1,2,4-oxadiazol-5-yl]-3′,4-dimethoxy-5′-methyl-spiro[benzofuran-2,4′-cyclohex-2-ene]-1′,3-dione;
(2S,5′R)-7-chloro-3′,4-dimethoxy-5′-methyl-6-(1H-pyrazol-5-yl) spiro[benzofuran-2,4′-cyclohex-2-ene]-1′,3-dione;
(2S,5′R)-7-chloro-3′,4-dimethoxy-6-[1-(2-methoxyethyl) pyrazol-3-yl]-5′-methyl-spiro[benzofuran-2,4′-cyclohex-2-ene]-1′,3-dione;
(2S,5′R)-7-chloro-6-(1,8-dioxa-2-azaspiro[4.5]dec-2-en-3-yl)-3′,4-dimethoxy-5′-methyl-spiro[benzofuran-2,4′-cyclohex-2-ene]-1′,3-dione;
(2S,5′R)-7-chloro-3′,4-dimethoxy-5′-methyl-6-(8-methyl-1-oxa-2,8-diazaspiro[4.5] dec-2-en-3-yl) spiro[benzofuran-2,4′-cyclohex-2-ene]-1′,3-dione;
(2S,5′R)-7-chloro-3′,4-dimethoxy-6-(2-methoxypyrimidin-5-yl)-5′-methyl-spiro[benzofuran-2,4′-cyclohex-2-ene]-1′,3-dione;
(2S,5′R)-7-chloro-3′,4-dimethoxy-6-(6-methoxy-3-pyridyl)-5′-methyl-spiro[benzofuran-2,4′-cyclohex-2-ene]-1′,3-dione;
(2S,5′R)-7-chloro-3′,4-dimethoxy-5′-methyl-6-(3-pyridyl) spiro[benzofuran-2,4′-cyclohex-2-ene]-1′,3-dione; or
(2S,5′R)-7-chloro-3′,4,6-trimethoxy-5′-methyl-spiro[benzofuran-2,4′-cyclohex-2-ene]-1′,3-dione.

26. (canceled)

27. The method of claim 25, wherein the compound is (2S,5′R)-7-chloro-3′,4-dimethoxy-5′-methyl-6-(5-methyl-1,3,4-oxadiazol-2-yl) spiro[benzofuran-2,4′-cyclohex-2-ene]-1′,3-dione or a pharmacologically acceptable salt thereof.

28. (canceled)

29. (canceled)

30. (canceled)
